(19) United States
(12) Patent Application Publication
(10) Pub. No.: US 2007/0059696 A1
(43) Pub. Date: Mar. 15, 2007

(54) Assessment method

(76) Inventors: Sandra Ebert, Gottingen (DE); Michael Uwe, Gottingen (DE); Roland Nau, Bovenden-Eddigehausen (DE); David Morritz De Kretser, Victoria (AU); David James Phillips, Victoria (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/549,557

(22) PCT Filed: Mar. 19, 2004
(86) PCT No.: PCT/AU04/00350

§ 371 Date: Oct. 2, 2006

(30) Foreign Application Priority Data
Mar. 19, 2003 (AU) 2003901267

(51) Int. Cl. C12Q 1/68; G01N 33/53

(52) U.S. Cl. 435/6; 435/7.1

(57) ABSTRACT

The present invention relates generally to a method of diagnosing, predicting and/or monitoring the development or progress of an inflammatory response in a mammal. More particularly, the present invention relates to a method of diagnosing, predicting and/or monitoring the development or progress of an inflammatory response by analysing one or both of activin or follistatin expression levels either in a subject mammal or in a biological sample derived from said mammal. The present invention further provides a method for predicting, diagnosing and/or monitoring conditions associated with or characterised by the onset of inflammatory response. Also provided are diagnostic agents useful for detecting activin and/or follistatin expression levels.

### FIELD OF THE INVENTION

The present invention relates generally to a method of diagnosing, predicting and/or monitoring the development or progress of an inflammatory response in a mammal. More particularly, the present invention relates to a method of diagnosing, predicting and/or monitoring the development or progress of an inflammatory response by analysing one or both of activin or follistatin expression levels either in a subject mammal or in a biological sample derived from said mammal. The present invention further provides a method for predicting, diagnosing and/or monitoring conditions associated with or characterised by the onset of an inflammatory response. Also provided are diagnostic agents useful for detecting activin and/or follistatin expression levels.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Mammals are required to defend themselves against a multitude of pathogens including viruses, bacteria, fungi and parasites, as well as non-pathogenic insults such as tumours and toxic, or otherwise harmful, agents. In response, effector mechanisms have evolved which are capable of mounting a defence against such antigens. These mechanisms are mediated by soluble molecules and/or by cells.

In the context of these effector mechanisms, inflammation is a complex multifaceted response to disease or injury which is regulated by the release of a cascade of cytokines.

These cytokines are classified in general terms as pro- or anti-inflammatory cytokines and the critical balance between release and activity of cytokines with opposing actions regulates the inflammatory response to prevent it from becoming overt or understated. If the inflammatory response continues unchecked and is overt then the host may suffer associated tissue damage. Conversely, a poor or understated inflammatory response may mean uncontrolled infection resulting in chronic illness and host damage. Regulation of the inflammatory response is important at both the systemic level and the local level.

The discovery of the detailed processes of inflammation has revealed a close relationship between inflammation and the immune response. There are five basic indicators of inflammation, these being redness (rubor), swelling (tumour), heat (calor), pain (dolor) and deranged function (finctio laesa). These indicators occur due to extravasation of plasma and infiltration of leukocytes into the site of inflammation. Consistent with these indicators, the main characteristics of the inflammatory response are therefore:
(i) vasodilation—widening of the blood vessels to increase the blood flow to the infected area;
(ii) increased vascular permeability—this allows diffusible components to enter the site;
(iii) cellular infiltration—this being the directed movement of inflammatory cells through the walls of blood vessels into the site of injury;
(iv) changes in biosynthetic, metabolic and catabolic profiles of many organs; and
(v) activation of cells of the immune system as well as of complex enzymatic systems of blood plasma.

The degree to which these characteristics occur is generally proportional to the severity of the injury and/or the extent of infection.

The inflammatory response can be broadly categorised into several phases. The earliest, gross event of an inflammatory response is temporary vasoconstriction, i.e. narrowing of blood vessels caused by contraction of smooth muscle in the vessel walls, which can be seen as blanching (whitening) of the skin. This is followed by several phases that occur over minutes, hours and days later, as follows:
(i) The acute vascular response follows within seconds of a tissue insult and lasts for some minutes. It is characterised by vasodilation and increased capillary permeability due to alterations in the vascular endothelium, leading to increased blood flow (hyperaemia) that causes redness (erythema) and the entry of fluid into the tissues (oedema).
(ii) If there has been sufficient damage to the tissues, or if infection has occurred, the acute cellular response takes place over the next few hours. The hallmark of this phase is the appearance of granulocytes, particularly neutrophils, in the tissue. These cells first attach themselves to the endothelial cells within the blood vessels (margination) and then cross into the surrounding tissue (diapedesis). If the vessel is damaged, fibrinogen and fibronectin are deposited at the site of injury, platelets aggregate and become activated and clot formation occurs.
(iii) If damage is sufficiently severe, a chronic cellular response may follow over the next few days. A characteristic of this phase of inflammation is the appearance of a mononuclear cell infiltrate composed of macrophages and lymphocytes. The macrophages are involved in microbial killing, in clearing up cellular and tissue debris, and are also thought to play a significant role in remodelling tissue.
(iv) Over the next few weeks, resolution may occur wherein normal tissue architecture is restored. Blood clots are removed by fibrinolysis. If it is not possible to return the tissue to its original form, scarring may occur from in-filling with fibroblasts, collagen, and new endothelial cells. Generally, by this time any infection will have been overcome, although this is not always the case and may result in further immunological responses, such as granuloma formation.

Inflammation is often considered in terms of acute inflammation that includes all the events of the acute vascular and acute cellular response (1 and 2 above), and chronic inflammation that includes the events during the chronic cellular response and resolution or scarring (3 and 4).

It should be understood, however, that in addition to the occurrence of inflammatory responses in a localised fashion in tissue which is damaged, infected or subject to an autoimmune response, for example, inflammatory responses may also occur systemically, such as in the case with sepsis.

In relation to sepsis, in particular, this condition is a major cause of morbidity and mortality worldwide and is the leading non-coronary cause of death in intensive care units. More than 700,000 cases of severe sepsis occur in the US annually at a healthcare cost of $17 billion annually.

Intense interest has focussed on the ability to discriminate between those patients who will die from sepsis and those who will survive. A number of diagnostic tests including body temperature, leukocyte count and various blood markers such as C-reactive protein, procalcitonin and various cytokines have been evaluated. While a number of these show predictive value in discriminating patient outcome, there is a need to continue to evaluate new markers or combinations of markers to improve diagnostic accuracy.

Accordingly, in light of the wide-ranging impact of inflammatory responses, there is an ongoing need to elucidate the complex mechanisms by which it functions. By identifying these mechanisms there is thereby provided scope for developing means of appropriately diagnosing, monitoring and/or treating inflammatory responses.

Inhibin, activin, and follistatin are three families of polypeptides originally isolated and characterized from ovarian follicular fluid based on their modulation of follicle stimulating hormone release from pituitary cell culture. In addition to their effects on follicle stimulating hormone synthesis and secretion, inhibin and activin have other biological functions. By contrast, the physiological significance of follistatin was obscure, until it was discovered that follistatin is a binding protein to activin.

Activins, composed of two β-subunits, β_{A}, β_{B}, β_{C}, β_{D}, and/or β_{E} are members of the transforming growth factor (TGF)-β superfamily [Vale et al; 1990, Handbook of Experimental Physiology, Vol. 95, Eds. Sporn & Roberts, Springer-Verlag, Berlin pp211-248]. Multimeric protein forms of activin include the homodimeric forms (Activin A—β_{A}β_{A}, Activin B—β_{B}β_{B}, Activin C—β_{C}β_{C}, Activin D—β_{D}β_{D}, and Activin E—β_{E}β_{E}) and the heterodimeric forms (for example, Activin AB—β_{A}β_{B}, Activin AC—β_{A}β_{C}, Activin AD—β_{A}β_{D}, or Activin AE—β_{A}β_{E}). The activins are multifunctional proteins. For example, Activin A, although originally identified as a regulator of follicle stimulating hormone release, is now known to exhibit the pleiotropic range of functional activities which are characteristic of most cytokines.

Follistatin functions as a biological regulator of activin. In fact, it was originally identified as an activin-binding protein. Follistatin is a monomeric protein which binds to activin with high affinity and is believed to thereafter lead to lysosomal degradation of the complexed activin.

Activin affects the growth and differentiation of many cell types, stimulates the secretion of follicle-stimulating hormone from the pituitary gland and inhibits growth hormone, prolactin, and adrenocorticotropin release [Billestrup et al., Molecular Endocrinology 1990 4 356-362; Kitaoka et al, Biochemical and Biophysical Research Communications 1988 157 48-54; Vale et al., Nature 1986 321 776-779]. Follistatin specifically binds to activin. As a result, circulating follistatin 315 neutralizes activin activity by preventing the interaction of the cytokine with its type II receptors [de Winter et al., Molecular and Cellular Endocrinology 1996 116 105-114] and, furthermore, cell surface-bound follistatin 288 facilitates the lysosomal degradation of activin [Hashimoto et al., Journal of Biological Chemistry 1997 272 13835-13842]. Both follistatin and activin mRNAs show a broad tissue distribution [Meunier et al., PNAS 1988 85 247-251; Michel et al., Biochemical and Biophysical Research Communications 1990 173 401-407; Schneider et al., European Journal of Endocrinology 2000 142 537-544]. Follistatin and activin are detectable in serum [Demura et al., Journal of Clinical Endocrinology and Metabolism 1993 76 1080-1082; Demura et al., Biochemical and Biophysical Research Communications 1992 185 1148-1154; Gilfillan et al., Clinical Endocrinology 1994 41 453-461; Khoury et al., Journal of Clinical Endocrinology and Metabolism 1995 80 1361-1368; Knight et al., Journal of Endocrinology 1996 148 267-279; McFarlane et al., European Journal ofEndocrinology 1996 134 481-489; Sakai et al., Biochemical and Biophysical Research Communications 1992 188 921-926; Sakamoto et al., European Journal of Endocrinology 1996 135 345-351; Tilbrook et al., Journal of Endocrinology 1996 149 55-63; Wakatsuki et aL, Journal of Clinical Endocrinology and Metabolism 1996 81 630-634], and their concentrations in serum increase with age [Wakatsuki et al. 1996, supra; Loria et al., European Journal of Endocrinology 1998 139 487-492]. At present, however, the precise sources of follistatin and activin in serum are unknown. Current data suggest that tissue-specific balances of follistatin and activin govern the growth and differentiation of responsive cell types in an autocrine/paracrine manner [Michel et al., Acta Endocrinologica 1993 129 525-531; Phillips et al., Trends in Endocrinology and Metabolism 2001 12 94-96].

An emerging roll for activin and follistatin in the body's innate immune response has been documented. For instance, activin and follistatin are secreted by various cell types in response to inflammatory compounds in vitro [Hübner et aL, Experimental Cell Research 1996 228 106-113; Jones et al., Endocrinology 2000 141 1905-1908; Keelan et aL, Placenta 2000 21 38-43; Michel et al., Endocrinology 1996 137 4925-4934; Phillips et al., Journal of Endocrinology 1998 156 77-82; Yu et al., Immunology 1996 88 368-374; Erämaa et al., Journal of Experimental Medicine 1992 176 1449-1452; Shao et al., Cytokine 1998 10 227-235; Mohan etal., European Journal of Endocrinology 2001 145 505-511]. Moreover, in some examples of inflammatory processes such as wound healing, inflammatory bowel disease and rheumatoid arthritis, increased activin and/or follistatin expression has been noted [Hübner et al., Laboratory Investigation 1997 77 311-318; Hübner et al., Developmental Biology 1996 173 490-498; Yu et al., Clinical and Experimental Immunology 1998 112 126-132]. However, since these very early and preliminary findings, the role of activin and follistatin in the context of inflammation, per se, has not been further elucidated, either in the context of their precise activities or in the context of the scope of the inflammatory conditions in which they function. In light of the extreme diversity in terms of the nature and extent of inflammatory responses which can occur, and the extremely pleiotropic activities of cytokines such as activin, it is not surprising that the preliminary findings of the mid to late 1990's have not progressed to more substantial theories. In particular, activin A and follistatin are expressed by a wide variety of cell types and most organs in the body in response to a wide range of stimuli. Accordingly, their usefulness as a marker of inflammation would therefore not be expected.

In sheep models of acute inflammatory challenge, activin and follistatin have been found to be elevated in the blood. However, until now there has been no human data to suggest that activin or follistatin are useful predictors of clinically important inflammatory diseases such as sepsis. While some workers have looked at levels of for instance, follistatin, in inflammatory conditions there has been no recognition until now that examination of those levels can provide useful information of the management of patients with inflammatory conditions. Thus for instance Michel et al, supra, although demonstrating that follistatin is elevated in septicemia, did not find a useful correlation with outcome/prognosis. Further, Michel et al, supra, demonstrated that follistatin is elevated in meningitis but did not find that this was correlated directly as a clinical indicator.

In work leading up to the present invention it has been surprisingly determined that activin and follistatin are in fact accurate and reliable diagnostic/prognostic indicators of the onset and severity of an inflammatory response. Accordingly, although some workers have observed increases in follistatin and/or activin in some inflammatory states, there has been no recognition that these levels in fact correlate to an accurate and reliable indicator of the predisposition to or onset of an inflammatory response and, more particularly, its likely severity. These findings have now facilitated the development of assessment technology directed to diagnosing, prognosing and/or monitoring the onset and/or severity of inflammatory responses or conditions characterised by an inflammatory response. This now provides means of effectively managing patients with inflammatory conditions.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention is directed to a method for detecting the onset or a predisposition to the onset of an inflammatory response in a mammal, said method comprising screening for the level of one or both of activin or follistatin protein and/or gene expression in said mammal wherein an increase in the level of said protein and/or gene expression is indicative of an inflammatory response.

Another aspect of the present invention is directed to a method of detecting the onset or a predisposition to the onset of an inflammatory response in a mammal, said method comprising screening for the level of one or both of activin A or follistatin protein and/or gene expression in said mammal wherein an increase in the level of said protein and/or gene expression is indicative of an inflammatory response.

In still another aspect there is provided a method of detecting the onset or a predisposition to the onset of a local inflammatory response in a mammal, said method comprising screening for the level of one or both of activin A or follistatin protein and/or gene expression wherein an increase in the level of said protein and/or gene expression is indicative of said local inflammatory response.

In yet another aspect there is provided a method of detecting the onset or a predisposition to the onset of a systemic inflammatory response in a mammal, said method comprising screening for the level of one or both of activin A or follistatin protein and/or gene expression wherein an increase in the level of said protein and/or gene expression is indicative of said systemic inflammatory response.

In still yet another aspect there is provided a method of detecting the onset or a predisposition to the onset of an acute systemic inflammatory response in a mammal, said method comprising screening for the level of one or both of activin A or follistatin protein and/or gene expression in a mammal wherein an increase in the level of said protein and/or gene expression is indicative of said acute systemic inflammatory response.

In yet still another aspect the present invention relates to a method for monitoring the progression of an inflammatory response in a mammal, said method comprising screening for modulation of the level of one or both of activin or follistatin protein and/or gene expression in said mammal.

A further aspect of the present invention provides a method for monitoring the progression of a localised inflammatory response in a mammal, said method comprising screening for modulation of the level of one or both of activin A or follistatin protein and/or gene expression in said mammal wherein an increase in the level of said protein and/or gene expression relative to a previously obtained level is indicative of the maintenance or worsening of said response and a decrease in said level is indicative of an improvement in said inflammatory response.

In another further aspect there is provided a method for monitoring the progression of a systemic inflammatory response in a mammal, said method comprising screening for modulation of the level of one or both of activin A or follistatin protein and/or gene expression relative to a previously obtained level wherein an increase in the level of said protein and/or gene expression in said mammal is indicative of the maintenance or worsening of said response and a decrease in said level is indicative of an improvement in said response.

In yet another further aspect, the present invention provides a method for assessing the severity of an inflammatory response in a mammal, said method comprising quantitatively screening for the level of one or both of activin or follistatin protein and/or gene expression in said mammal wherein the degree of increase in the level of said protein and/or gene expression is indicative of the severity of said inflammatory response.

In still another further aspect there is a provided a method for assessing the severity of an inflammatory response in a mammal, said method comprising quantitatively screening for the level of one or both of activin A or follistatin protein and/or gene expression in said mammal wherein the degree of increase in the level of said protein and/or gene expression is indicative of the severity of said inflammatory response.

Yet still another further aspect of the present invention is directed to a method for detecting the onset or a predisposition to the onset of a condition characterised by an inflammatory response in a mammal, said method comprising screening for the level of one or both of activin or follistatin protein and/or gene expression in said mammal where an increase in the level of said protein and/or gene expression is indicative of the onset or predisposition to the onset of said condition.

In still yet another further aspect there is provided a method for detecting the onset or a predisposition to the onset of a condition characterised by an inflammatory response in a mammal, said method comprising screening for the level of one or both of activin A or follistatin protein and/or gene expression in said mammal wherein an increase in the level of said protein and/or gene expression is indicative of the onset or a predisposition to the onset of said condition.

Yet another aspect is directed to a method for monitoring the progression of a condition characterised by an inflammatory response in a mammal, said method comprising screening for modulation of the level of one or both of activin or follistatin proteins and/or gene expression in said mammal wherein an increase in the level of said protein and/or gene expression relative to a previously obtained level is indicative of the maintenance or worsening of said condition and a decrease in said level is indicative of an improvement in said condition.

More particularly, there is provided a method for monitoring the progression of a condition characterised by an inflammatory response in a mammal, said method comprising screening for modulation of the level of one or both of activin A or follistatin proteins and/or gene expression in said mammal wherein an increase in the level of said protein and/or gene expression relative to a previously obtained level is indicative of the maintenance or worsening of said condition and a decrease in said level is indicative of an improvement in said condition.

In yet another aspect there is provided a method for assessing the severity of a condition characterised by an inflammatory response in a mammal, said method comprising quantitatively screening for the level of one or both of activin or follistatin protein and/or gene expression in said mammal wherein the degree of increase in the level of said protein and/or gene expression is indicative of the severity of said condition.

Another aspect of the present invention provides a diagnostic kit for assaying biological samples comprising an agent for detecting the marker proteins or encoding nucleic acid molecules and reagents useful for facilitating the detection by the agent in the first compartment. Further means may also be included, for example, to receive a biological sample. The agent may be any suitable detecting molecule.

### BRIEF DESCRIPTION OF THE DRAWINGS

FIG. 1 is a graphical representation of the time course of activin, follistatin and C-reactive protein concentrations in serum of female (A) and male (B) patients with septicemia. Patient numbers correspond to the numbers in Table 1. In each diagram on the X-axis, the time points of blood sampling are shown (first sample taken at 90 hr). On the left Y-axis, follistatin serum concentrations in ng/ml and on the right Y-axis activin serum concentrations in ng/ml are shown. The second Y-axis on the left is the scale of the C-reactive protein serum concentrations in ng/ml.

FIG. 2 is a graphical representation of a time course of activin, follistatin and C-reactive protein concentrations in serum of patent A.S. a 31 year old male diagnosed with Neisseria meningitidis meningitis and sepsis who subsequently died.

FIG. 3 is a graphical representation of a time course of activin, follistatin and C-reactive protein concentrations in serum of patient M.B. a 43 year old female diagnosed with gastroenteritis who recovered.

FIG. 4 is a graphical representation of a time course of activin, follistatin and C-reactive protein concentrations in serum of patient Ho a 48 year old male diagnosed with a cutaneous infection. The patient recovered.

FIG. 5 is a graphical representation of a time course of activin, follistatin and C-reactive protein concentrations in serum of patient M.F. a 29 year old female diagnosed with Staphylococcus aureus sepsis who recovered.

FIG. 6 is a graphical representation of a time course of activin, follistatin and C-reactive protein concentrations in serum of patient M.M. a 52 year old male diagnosed with cirrhosis who subsequently died.

FIG. 7 is a graphical representation of a time course of activin, follistatin and C-reactive protein concentrations in serum of patient U.D. a 33 year old male diagnosed with Streptococcus pneumoniae sepsis who subsequently died.

FIG. 8 is a graphical representation of a time course of activin, follistatin and C-reactive protein concentrations in serum of patient W/L. an 87 year old male diagnosed with pneumonia who subsequently died.

FIG. 9 is a graphical representation of a time course of activin, follistatin and C-reactive protein concentrations in serum of patient W.S. a 58 year old male diagnosed with intracranial bleeding.

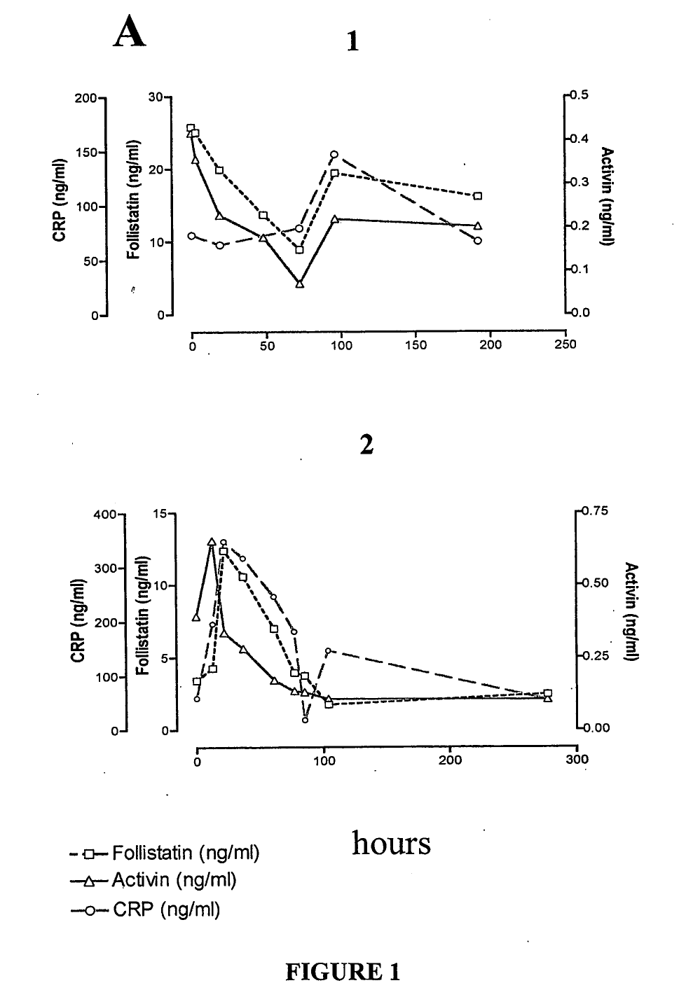

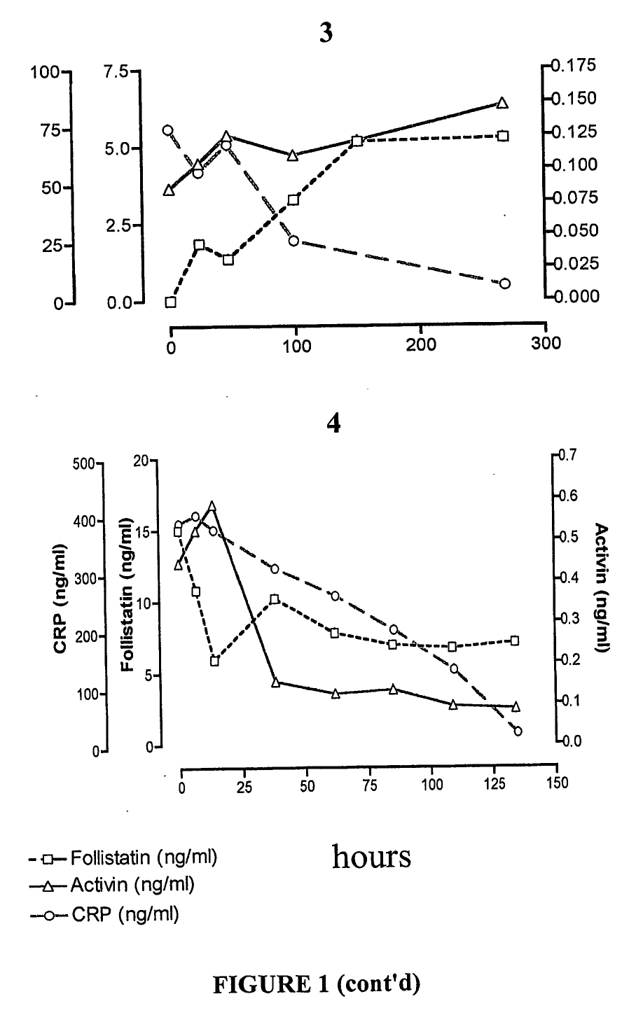

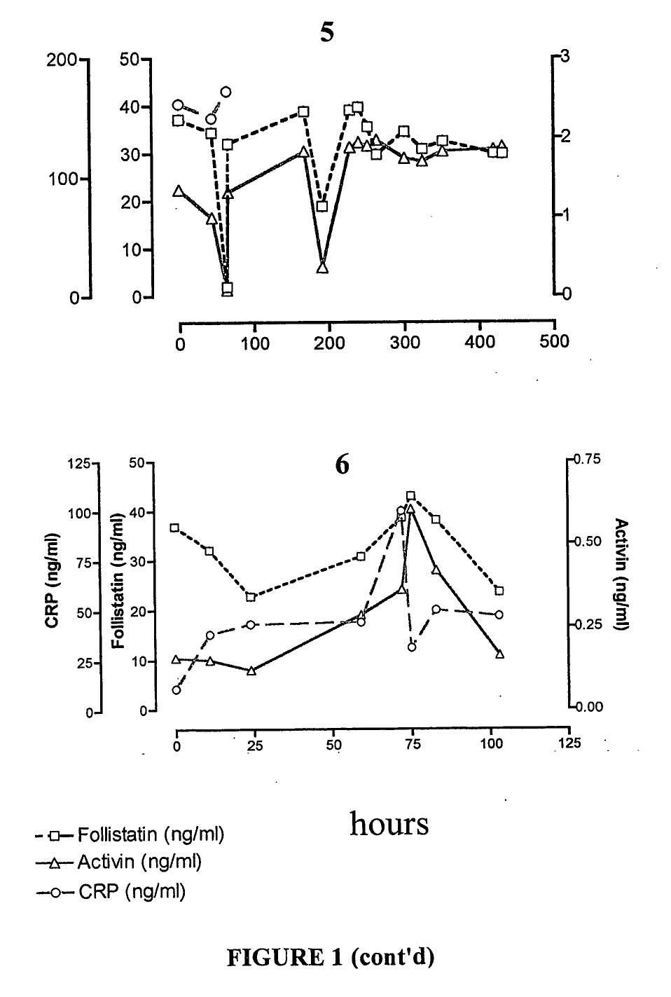

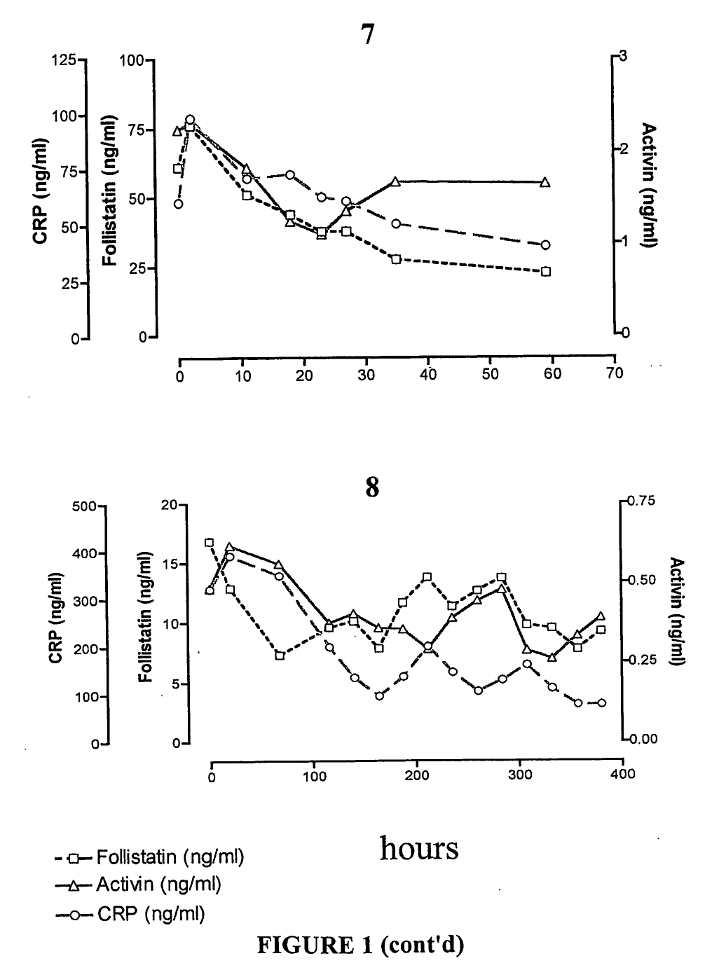

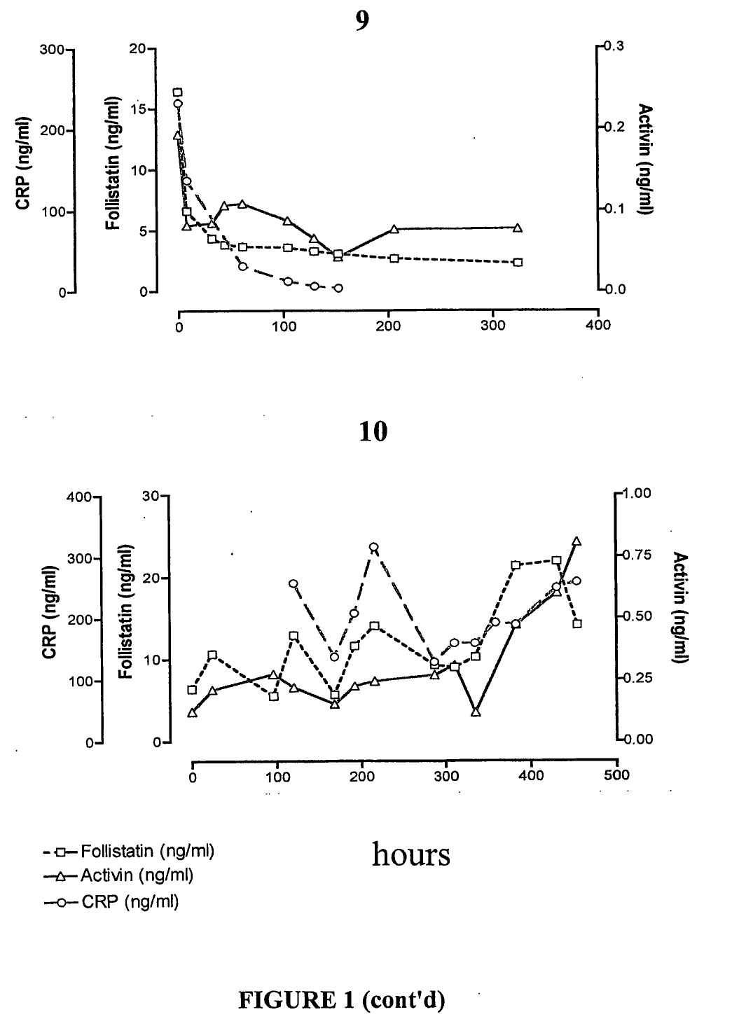

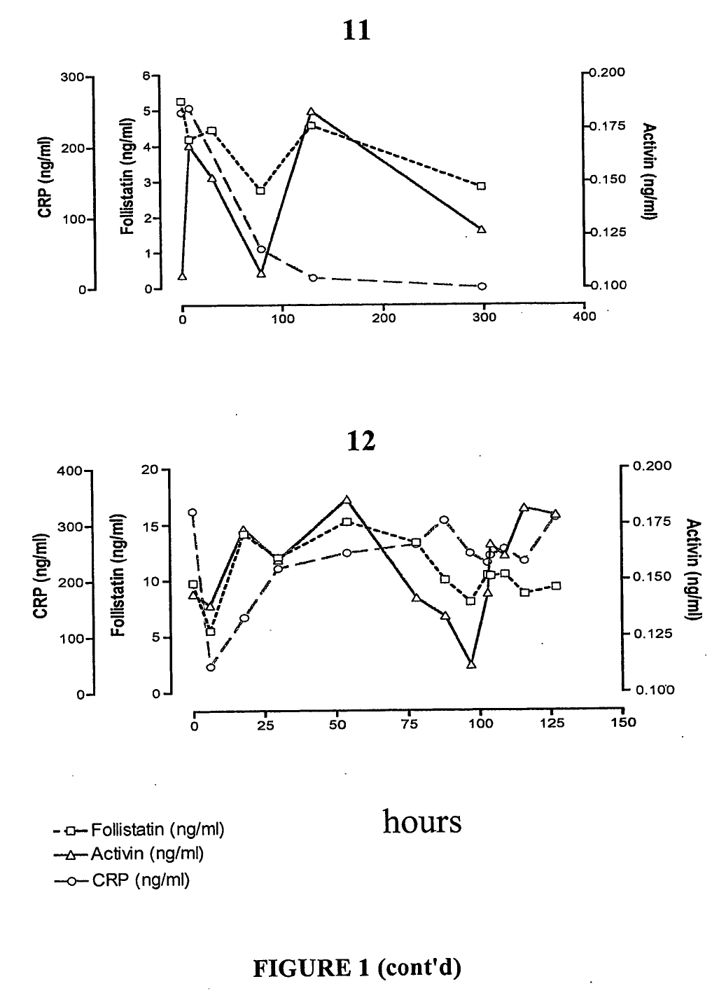

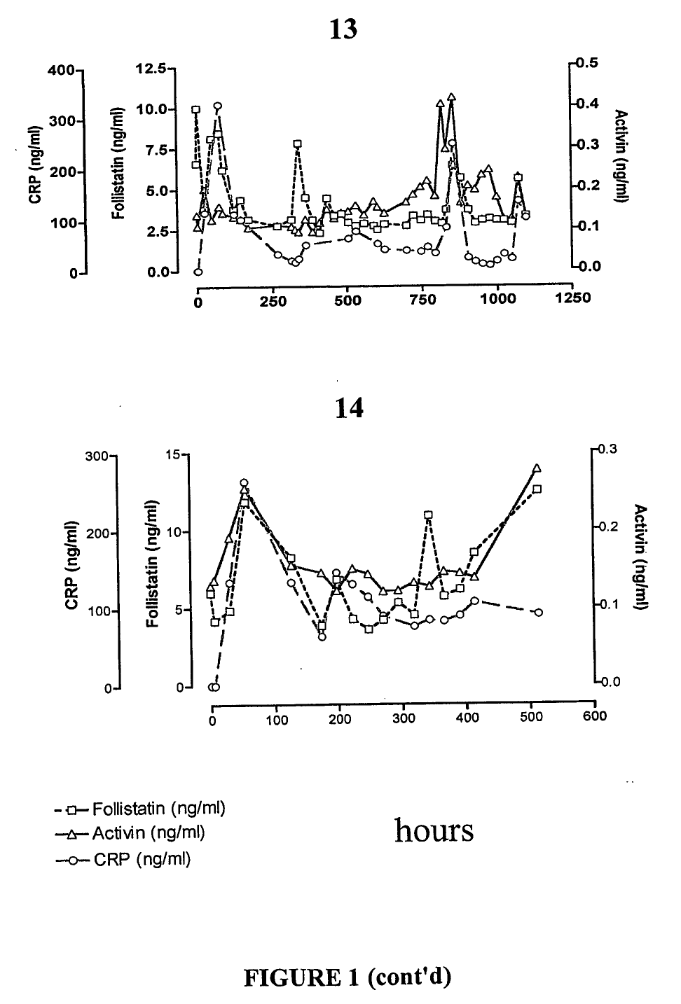

### DETAILED DESCRIPTION OF THE INVENTION

The present invention is predicated, in part, on the determination that activin and follistatin are accurate and highly sensitive indicators of both the onset or predisposition to the onset of an inflammatory response and the likely severity of such a response. In particular, the present invention provides a means of assessing a systemic inflammatory response based on relative systemic levels of activin and/or follistatin. These findings have therefore facilitated the development of a highly sensitive and informative assay directed to diagnosing the onset or predisposition to the onset of an inflammatory response or a condition characterised by an inflammatory response.

Accordingly, one aspect of the present invention is directed to a method for detecting the onset or a predisposition to the onset of an inflammatory response in a mammal, said method comprising screening for the level of one or both of activin or follistatin protein and/or gene expression in said mammal wherein an increase in the level of said protein and/or gene expression is indicative of an inflammatory response.

More particularly, the present invention is directed to a method of detecting the onset or a predisposition to the onset of an inflammatory response in a mammal, said method comprising screening for the level of one or both of activin A or follistatin protein and/or gene expression in said mammal wherein an increase in the level of said protein and/or gene expression is indicative of an inflammatory response.

Without limiting the present invention to any one theory or mode of action, the inflammatory response is a complex response characterised by a series of physiological and/or immunological events which are induced to occur by the release of a cytokine cascade in response to any one of a variety of stimuli including, but not limited to, tissue injury, infection, an immune response (such as to a pathogen or an innocuous agent—as occurs with allergies), or disease (such as tumour formation or an autoimmune response).

The physiological events which characterise inflammation include:
(i) vasodilation
(ii) increased vascular permeability
(iii) cellular infiltration
(iv) changes to the biosynthetic, metabolic and catabolic profiles of affected organs
(v) activation of the cells of the immune system.

It should be understood that reference to an “inflammatory response” is a reference to any one or more of the physiological and/or immunological events or phases that are induced to occur in the context of inflammation and, in general, in response to the signals generated by the cytokine cascade which largely directs an inflammatory response. For example IL-1, TNFα and IL-6 are well known for their functions as pro-inflammatory mediators. It should also be understood that an inflammatory response within the context of the present invention essentially includes a reference to a partial response, such as a response which has only just commenced, or to any specific phase or event of a response (such as the phases and events detailed in points (i)-(v), above, or any other effect related to inflammation including, but not limited to, the production of acute phase proteins—including complement components, fever and a systemic immune response). Further, it should also be understood that depending on any given set of specific circumstances, the end point of an inflammatory response may vary. For example, in some situations there may only occur an acute vascular response. To the extent that “acute” inflammation occurs, this is generally understood to include the events of both an acute vascular response and an acute cellular response. Some inflammatory responses will resolve at the acute stage while others may progress to become chronic cellular responses.

Without limiting the present invention to any one theory or mode of action, in certain circumstances the acute process, characterized by neutrophil infiltration and oedema, gives way to a predominance of mononuclear phagocytes and lymphocytes. This is thought to occur to some degree with the normal healing process but becomes exaggerated and chronic when there is ineffective elimination of foreign materials as in certain infections e.g. tuberculosis) or following introduction of foreign bodies (e.g. asbestos) or deposition of crystals (e.g. urate crystals). Chronic inflammation is often associated with fusion of mononuclear cells to form multinucleated gigant cells, which eventually become a granuloma. Chronic inflammation is also seen under conditions of delayed hypersensitivity. The subject inflammatory response may be systemic or localised. Examples of systemic inflammatory responses include those which fall within the scope of systemic inflammatory response syndrome such as septic shock, toxic shock or septicaemia.

Examples of localised inflammatory responses include those which occur in the context of rheumatoid arthritis, inflammatory bowel disease, pancreatitis, atherosclerosis, meningitis, appendicitis, angiogenesis, psoriasis, neural protection, renal tubular necrosis, allergic responses and wound healing (for example, pursuant to surgery, burns or other tissue injury). It should be understood, however, that some localised inflammatory responses can become systemic, for example as can occur when the onset of septic shock occurs as a complication of severe burns or abdominal wounds. In another example, septicaemia can result from the transition of a more localised bacterial infection to a circulatory infection.

Accordingly, in one preferred embodiment there is provided a method of detecting the onset or a predisposition to the onset of a local inflammatory response in a mammal, said method comprising screening for the level of one or both of activin A or follistatin protein and/or gene expression wherein an increase in the level of said protein and/or gene expression is indicative of said local inflammatory response.

More preferably, said local inflammatory response is acute.

In another preferred embodiment there is provided a method of detecting the onset or a predisposition to the onset of a systemic inflammatory response in a mammal, said method comprising screening for the level of one or both of activin A or follistatin protein and/or gene expression wherein an increase in the level of said protein and/or gene expression is indicative of said systemic inflammatory response.

More preferably, said systemic inflammatory response is acute.

According to this most preferred embodiment there is provided a method of detecting the onset or a predisposition to the onset of an acute systemic inflammatory response in a mammal, said method comprising screening for the level of one or both of activin A or follistatin protein and/or gene expression in a mammal wherein an increase in the level of said protein and/or gene expression is indicative of said acute systemic inflammatory response.

In accordance with these preferred aspects of the present invention, said acute inflammatory response occurs in the context of, or is otherwise associated with, septic shock, septicaemia, appendicitis, meningitis, hepatic response to toxins or viruses, angiogenesis, psoriasis, neural protection, atherosclerosis, renal tubular necrosis, or wound healing or traumatic injury such as occurs with surgery and burns.

Preferably, said acute systemic inflammatory response occurs in the context of systemic inflammatory response syndrome and even more particularly sepsis, septicaemia, toxic shock, septic shock, tissue trauma, meningitis or appendicitis.

Most preferably, in accordance with these aspects of the present invention activin A and follistatin are both screened for.

As detailed hereinbefore, the present invention is predicated on the determination that increases in the level of expression of activin A and/or follistatin is indicative of the onset or a predisposition to the onset of an inflammatory response.

Reference to “activin A” should be understood as a reference to all forms of activin A and to fragments, derivatives, mutants or variants thereof. Activin A is a dimeric protein which comprises two activin β_{A} monomers. It should also be understood to include reference to a dimer comprising any isoforms which may arise from alternative splicing of activin β_{A} mRNA or mutant or polymorphic forms of activin β_{A}. Reference to “activin A” should be understood to include reference to all forms of these molecules including all precursor, proprotein or intermediate forms thereof. Reference to activin A should also be understood to extend to any activin A protein, whether existing as a dimer, multimer or fusion protein. Accordingly, it should be understood that although one will preferably screen for the activin A dimer, one may also develop suitable screening methods based on detecting one or both of the activin β_{A} subunits, individually.

Reference to “follistatin” should be read as including reference to all forms of follistatin and to fragments, derivatives, mutants or variants thereof including, by way of example, the three protein cores and six molecular weight forms which have been identified as arising from the alternatively spliced mRNAs FS315 and FS288. Accordingly, it should also be understood to include reference to any isoforms which may arise from alternative splicing of follistatin mRNA or mutant or polymorphic forms of follistatin. It should still further be understood to extend to any protein encoded by the follistatin gene, any subunit polypeptide, such as precursor forms which may be generated, an any follistatin protein, whether existing as a monomer, multimer or fusion protein.

The term “mammal” as used herein includes humans, primates, livestock animals (eg. horses, cattle, sheep, pigs, donkeys), laboratory test animals (eg. mice, rats, guinea pigs), companion animals (eg. dogs, cats) and captive wild animal (eg. kangaroos, deer, foxes). Preferably, the mammal is a human or a laboratory test animal. Even more preferably, the mammal is a human.

The present invention is predicated on the determination that activin A and follistatin expression levels become increased in the context of an inflammatory response. Without limiting the present invention to any one theory or mode of action, it has been determined that within minutes of an inflammatory stimulus, activin A levels are increased. This is followed by the release of a cascade of cytokines including TNFα, IL-6 and follistatin. Accordingly, activin A is one of the earliest cytokines released subsequently to an initial inflammatory response stimulus and may, in fact, initiate the entire inflammatory cascade. The method of the present invention can therefore detect both the onset of an inflammatory response and, to the extent that inflammation-related symptoms are not yet evident, a predisposition to the development of an inflammatory response since the upregulation of one or both of activin A and follistatin is indicative of the forthcoming development of one or more phases or events of an inflammatory response. Reference to “detecting” an inflammatory response should therefore be understood in its broadest context and includes, inter alia, diagnosing, screening, confirming or otherwise assessing an inflammatory response or a condition characterised by the onset of an inflammatory response.

The method of the present invention is predicated on the correlation of activin A and/or follistatin in individuals with normal levels of these molecules. The “normal level” is the level of activin A and/or follistatin in a corresponding biological sample of a subject who has not developed an inflammatory response nor is predisposed to the development of an inflammatory response in the context described above. Without limiting the present invention in any way, it is generally believed that the systemic level of activin A and/or follistatin, to the extent that one is screening at the systemic level, in a normal individual will be negligible.

Accordingly, the term “modulation” refers to increases and decreases in activin A and/or follistatin levels relative either to a normal reference level (or normal reference level range) or to an earlier activin A or follistatin level result determined from the subject. A normal reference level is the activin A and/or follistatin level from a relevant biological sample of a subject or group of subjects which are not experiencing an inflammatory response. In a preferred embodiment, said normal reference level is the level determined from one or more subjects of a relevant cohort to that of the subject being screened by the method of the invention. By “relevant cohort” is meant a cohort characterised by one or more features which are also characteristic of the subject who is the subject of screening. These features include, but are not limited to, age, gender, ethnicity or health status, for example.

This reference level may be a discrete figure or may be a range of figures. The reference level may vary between individual classes of activin A and/or follistatin molecules (such as the differentially spliced forms of follistatin).

Although the preferred method is to detect an increase in activin A and/or follistatin levels in order to diagnose the onset or a predisposition to the onset of an inflammatory response, the detection of a decrease in the levels of these molecules may be desired under certain circumstances. For example, to monitor improvement in the status of an inflammatory response during the course of prophylactic or therapeutic treatment of patients presenting with an acute or chronic inflammatory response or a condition associated with such a response, such as sepsis, septicaemia, meningitis, rheumatoid arthritis, or a tissue trauma. Further, although upregulation in the levels of these molecules will generally be regarded as adverse, since it is likely to be indicative of an unwanted inflammatory response, in some situations one may be screening for the induction of a desired inflammatory response such as where an inflammatory response is designed to provide adjuvant-like activity. This may be particularly useful in the context of anti-tumour therapy. In still another example, the upregulation of host defence mechanisms may be desired.

This aspect of the present invention also enables one to monitor the progression of an inflammatory response or a condition characterised by an inflammatory response. By “progression” is meant the ongoing nature of an inflammatory response, such as its improvement, maintenance, worsening or a change in the level of its severity.

Accordingly, another aspect of the present invention relates to a method for monitoring the progression of an inflammatory response in a mammal, said method comprising screening for modulation of the level of one or both of activin or follistatin protein and/or gene expression in said mammal.

Preferably, said activin is activin A.

In one particularly preferred embodiment, said inflammatory response is an acute localised inflammatory response or an acute systemic inflammatory response.

In accordance with these preferred aspects of the present invention, said acute inflammatory responses occurs in the context of, or is otherwise associated with, septic shock, septicaemia, appendicitis, meningitis, hepatic response to toxins or viruses, angiogenesis, psoriasis, neural protection, atherosclerosis, renal tubular necrosis, or wound healing or traumatic injury such as occurs with surgery and burns.

Most preferably, said acute systemic inflammatory response occurs in the context of systemic inflammatory response syndrome and even more particularly sepsis, toxic shock, septic shock, septicaemia, tissue trauma, meningitis or appendicitis.

Most preferably, in accordance with these aspects of the present invention activin A and follistatin are both screened for.

It should be understood that in accordance with this aspect of the present invention, activin A and/or follistatin levels will likely be assessed relative to one or more previously obtained levels from the patient in issue.

One particularly preferred embodiment of the present invention therefore provides a method for monitoring the progression of a localised inflammatory response in a mammal, said method comprising screening for modulation of the level of one or both of activin A or follistatin protein and/or gene expression in said mammal wherein an increase in the level of said protein and/or gene expression relative to a previously obtained level is indicative of the maintenance or worsening of said response and a decrease in said level is indicative of an improvement in said inflammatory response.

Preferably, said inflammatory response is an acute response.

In another particularly preferred embodiment there is provided a method for monitoring the progression of a systemic inflammatory response in a mammal, said method comprising screening for modulation of the level of one or both of activin A or follistatin protein and/or gene expression relative to a previously obtained level wherein an increase in the level of said protein and/or gene expression in said mammal is indicative of the maintenance or worsening of said response and a decrease in said level is indicative of an improvement in said response.

Preferably, said inflammatory response is a systemic response.

Most preferably, in accordance with these aspects of the present invention activin A and follistatin are both screened for.

The inventors have still further determined that a correlation exists in relation to the quantitative level of activin A and/or follistatin which is observed in a patient and the severity of an inflammatory response. In this regard, the severity of such a response correlates to likely patient outcome, such as patient survival. Such clinical information is extremely valuable since it can provide the basis upon which a therapeutic or palliative treatment regime is based or modified. For example, in those patients assessed as exhibiting a likely poor outcome, more aggressive therapeutic treatments can be initiated.

Specifically, it has been determined that the higher the level of activin A and/or follistatin expression, the more severe is the inflammatory response and therefore the poorer the likely outcome for the patient in issue. Accordingly, the present invention provides a means of both diagnosing and monitoring the existence of an inflammatory response in a qualitative manner and also assessing the severity of the response in a patient at a given point in time. In one particular aspect, it has been determined that activin A and/or follistatin over a defined level is predictive of death. In the context of the monitoring of a patient, this is an extremely valuable tool.

Accordingly, in yet another aspect, the present invention provides a method for assessing the severity of an inflammatory response in a mammal, said method comprising quantitatively screening for the level of one or both of activin or follistatin protein and/or gene expression in said mammal wherein the degree of increase in the level of said protein and/or gene expression is indicative of the severity of said inflammatory response.

More particularly, there is a provided a method for assessing the severity of an inflammatory response in a mammal, said method comprising quantitatively screening for the level of one or both of activin A or follistatin protein and/or gene expression in said mammal wherein the degree of increase in the level of said protein and/or gene expression is indicative of the severity of said inflammatory response.

Preferably, said inflammatory response is an acute localised inflammatory response or an acute localised response.

In accordance with these preferred aspects of the present invention, said acute inflammatory responses occur in the context of, or is otherwise associated with, septic shock, toxic shock, sepsis, septicaemia, appendicitis, pancreatitis, meningitis, hepatic response to toxins or viruses, angiogenesis, psoriasis, neural protection, atherosclerosis, renal tubular necrosis, or wound healing or traumatic injury such as occurs with surgery and burns.

Most preferably, said acute systemic inflammatory response occurs in the context of systemic inflammatory response syndrome and even more particularly sepsis, toxic shock, septic shock, septicaemia, tissue trauma, meningitis or appendicitis.

Most preferably, in accordance with these aspects of the present invention activin A and follistatin are both screened for.

To the extent that said acute systemic inflammatory response is related to sepsis and the biological sample which is the subject of analysis is a blood product sample, a level of activin A and/or follistatin at least about 2 times higher than levels within the normal range is indicative of poor prognosis. More particularly for a patient with sepsis a level of activin A and/or follistatin at least about 3 times higher than levels within the normal range is indicative of poor prognosis.

In one particular embodiment of the invention, the level of activin and/or follistatin functions as a predictor of possible death when the activin A level is greater than 0.3 ng/ml over a 24 hour period and/or the level of follistatin is greater than 20 ng/ml over a 24 hour period when measured by assays as herein described. In this regard, the assessment of level in the context of a time period minimizes possible difficulties associated with the fact that patients being assessed will be at different stages of disease.

Most particularly, said poor prognosis correlates to death.

The method of the present invention has widespread application including, but not limited to the diagnostic/prognostic analysis of an inflammatory response or inflammatory response symptoms or aspects of any condition characterised by the presence of an inflammatory response such as septic shock, septicaemia, appendicitis, meningitis, hepatic response to toxins or viruses, angiogenesis, psoriasis, neural protection, atherosclerosis, renal tubular necrosis, or wound healing or traumatic injury such as occurs with surgery and burns.

Accordingly, another aspect of the present invention is directed to a method for detecting the onset or a predisposition to the onset of a condition characterised by an inflammatory response in a mammal, said method comprising screening for the level of one or both of activin or follistatin protein and/or gene expression in said mammal where an increase in the level of said protein and/or gene expression is indicative of the onset or predisposition to the onset of said condition.

More particularly, there is provided a method for detecting the onset or a predisposition to the onset of a condition characterised by an inflammatory response in a mammal, said method comprising screening for the level of one or both of activin A or follistatin protein and/or gene expression in said mammal wherein an increase in the level of said protein and/or gene expression is indicative of the onset or a predisposition to the onset of said condition.

Yet another aspect is directed to a method for monitoring the progression of a condition characterised by an inflammatory response in a mammal, said method comprising screening for modulation of the level of one or both of activin or follistatin proteins and/or gene expression in said mammal wherein an increase in the level of said protein and/or gene expression relative to a previously obtained level is indicative of the maintenance or worsening of said condition and a decrease in said level is indicative of an improvement in said condition.

More particularly, there is provided a method for monitoring the progression of a condition characterised by an inflammatory response in a mammal, said method comprising screening for modulation of the level of one or both of activin A or follistatin proteins and/or gene expression in said mammal wherein an increase in the level of said protein and/or gene expression relative to a previously obtained level is indicative of the maintenance or worsening of said condition and a decrease in said level is indicative of an improvement in said condition.

In yet another aspect there is provided a method for assessing the severity of a condition characterised by an inflammatory response in a mammal, said method comprising quantitatively screening for the level of one or both of activin or follistatin protein and/or gene expression in said mammal wherein the degree of increase in the level of said protein and/or gene expression is indicative of the severity of said condition.

Preferably, said activin is activin A.

In accordance with these aspects of the present invention, said inflammatory response is preferably an acute localised inflammatory response or an acute localised response.

In accordance with these preferred aspects of the present invention, said acute inflammatory responses occur in the context of, or is otherwise associated with, septic shock, toxic shock, sepsis, septicaemia, appendicitis, pancreatitis, meningitis, hepatic response to toxins or viruses, angiogenesis, psoriasis, neural protection, atherosclerosis, renal tubular necrosis, or wound healing or traumatic injury such as occurs with surgery and burns.

Most preferably, said acute systemic inflammatory response occurs in the context of systemic inflammatory response syndrome and even more particularly sepsis, septic shock, toxic shock, septicaemia, tissue trauma, meningitis or appendicitis.

Most preferably, in accordance with these aspects of the present invention activin A and follistatin are both screened for.

It should be understood that the screening methodology herein defined may be performed either quantitatively or qualitatively. Although it is likely that quantitative analyses will be preferred since they provide information in relation to both the existence, or not, of an inflammatory condition in addition to identifying its severity, the method of the present invention does facilitate qualitative analyses. In particular, since activin A and follistatin are usually not found in the blood in appreciable amounts, to the extent that systemic analysis is being performed a test directed to assessing the presence or not of activin and/or follistatin will provide useful information. It will also provide scope for establishing extremely simple and inexpensive screening procedures.

Methods of screening for levels of activin and/or follistatin can be achieved by any suitable method which would be well known to persons of skill in the art. In this regard, it should be understood that reference to screening for the level of protein and/or gene expression “in a mammal” is intended as a reference to the use of any suitable technique which will provide information in relation to the level of expression of activin and/or follistatin in the relevant tissue of the mammal. These screening techniques include both in vivo screening techniques, as hereinafter described, as well as the in vitro techniques which are applied to a biological sample extracted from said mammal. Such in vitro techniques are likely to be preferred due to their significantly more simplistic and routine nature.

Since the present invention is predicated on screening for changes in the level of activin A and/or follistatin proteins, such changes can in fact be screened for at the protein level or at the nucleic acid level, such as by screening for increases in the level of activin A and/or follistatin mRNA transcripts. The person of skill in the art will determine the most appropriate means of analysis in any given situation. However it is generally preferred that screening be performed in the context of protein molecules due to the relative simplicity of the techniques which are likely to be utilized. Nevertheless in certain situations, and in the context of particular biological samples, it may be desirable or otherwise useful to directly analyze gene transcription.

Still further, to the extent that one is analysing a biological sample harvested from a patient, it is within the skill of the person in the art to determine the most appropriate sample for analysis. For example, blood components are likely to provide a most convenient means for analysing systemic levels of activin A and/or follistatin protein levels in the context of systemic inflammatory responses. However, to the extent that one is assessing potential localised inflammatory responses, other types of biological samples may be more suitable. For example, early stage rheumatoid arthritis may be initially assessed by determining the presence or level of inflammatory response by analysing levels of activin A and/or follistatin in the synovial fluid of an affectedjoint. Similarly, suspected meningitis may be assessed in terms of the degree of inflammatory response by analysing the spinal fluid which is generally automatically harvested from a patient for the purpose of a range of routine analytical tests which are performed. In other situations, it may be more appropriate to analyze biopsy specimens.

Reference to a “biological sample” should therefore be understood as a reference to any sample of biological material derived from an individual such as, but not limited to, mucus, stool, urine, blood, serum, cell extract, biopsy specimens and fluid which has been introduced into the body of an individual and subsequently removed such as, for example, the saline solution extracted from the lung following lung lavage or the solution retrieved from an enema wash. The biological sample which is tested according to the method of the present invention may be tested directly or may require some form of treatment prior to testing. For example, a biopsy sample may require homogenisation or sectioning prior to testing.

In a preferred embodiment, the subject inflammatory response which is under investigation is a systemic inflammatory response and the biological sample which is subjected to analysis is a blood sample, or a component of a blood sample. Most preferably, the protein forms of activin A and follistatin are screened for.

As described above, means of screening for changes in activin A and/or follistatin (herein referred to as “the markers”) levels in an individual, or biological sample derived therefrom, can be achieved by any suitable method, which would be well known to the person of skill in the art, such as but not limited to:
(i) In vivo detection of the markers. Molecular Imaging may be used following administration of imaging probes or reagents capable of disclosing altered expression levels of the markers mRNA or protein expression product in the prostate tissues.
   Molecular imaging (Moore, A., Basilion, J., Chiocca, E., and Weissleder, R., BBA, 1402:239-249, 1988; Weissleder, R., Moore, A., Ph.D., Mahmood-Bhorade, U., Benveniste, H., Chiocca, E. A., Basilion, J. P. Nature Medicine, 6:351-355, 2000) is the in vivo imaging of molecular expression that correlates with the macro-features currently visualized using “classical” diagnostic imaging techniques such as X-Ray, computed tomography (CT), MRI, Positron Emission Tomography (PET) or endoscopy. Historically, detection of malignant tumor cells in a background of normal or hyperplastic benign tissue is often based on differences in physical properties between tissues, which are frequently minimal, resulting in low contrast resolution. Application of expression profiling will define the differences in “molecular properties” between cancer and normal tissues that arise as a result of malignant transformation.
(ii) Detection of up-regulation of mRNA expression in the cells by Fluorescent In Situ Hybridization (FISH), or in extracts from the cells by technologies such as Quantitative Reverse Transcriptase Polymerase Chain Reaction (QRTPCR) or Flow cytometric qualification of competitive RT-PCR products (Wedemeyer, N., Potter, T., Wetzlich, S. and Gohde, W. Clinical Chemistry 48:9 1398-1405, 2002) or array technologies.
   For example, a labelled polynucleotide encoding the markers may be utilized as a probe in a Northern blot of an RNA extract obtained from the prostate. Preferably, a nucleic acid extract from the animal is utilized in concert with oligonucleotide primers corresponding to sense and antisense sequences of a polynucleotide encoding the markers, or flanking sequences thereof, in a nucleic acid amplification reaction such as RT PCR, real time PCR or SAGE. A variety of automated solid-phase detection techniques are also appropriate. For example, a very large scale immobilized primer arrays (VLSIPS™ are used for the detection of nucleic acids as, for example, described by Fodor et al., 1991 and Kazal et al., 1996. The above genetic techniques are well known to persons skilled in the art.
   For example, to detect the markers encoding RNA transcripts, RNA is isolated from a cellular sample suspected of containing the markers RNA, e.g. total RNA isolated from human prostate cancer tissue. RNA can be isolated by methods known in the art, e.g. using TRIZOL™ reagent (GIBCO-BRL/Life Technologies, Gaithersburg, Md.). Oligo-dT, or random-sequence oligonucleotides, as well as sequence-specific oligonucleotides can be employed as a primer in a reverse transcriptase reaction to prepare first-strand cDNAs from the isolated RNA. Resultant first-strand cDNAs are then amplified with sequence-specific oligonucleotides in PCR reactions to yield an amplified product.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers. These primers will be identical or similar in sequence to opposite strands of the template to be amplified. PCR can be used to amplify specific RNA sequences and cDNA transcribed from total cellular RNA. See generally Mullis et al., 1987; Erlich, 1989. Thus, amplification of specific nucleic acid sequences by PCR relies upon oligonucleotides or “primers” having conserved nucleotide sequences wherein the conserved sequences are deduced from alignments of related gene or protein sequences, e.g. a sequence comparison of mammalian the markers genes. For example, one primer is prepared which is predicted to anneal to the antisense strand and another primer prepared which is predicted to anneal to the sense strand of a cDNA molecule which encodes the markers.
To detect the amplified product, the reaction mixture is typically subjected to agarose gel electrophoresis or other convenient separation technique and the relative presence of the markers specific amplified DNA detected. For example, the markers amplified DNA may be detected using Southern hybridization with a specific oligonucleotide probe or comparing is electrophoretic mobility with DNA standards of known molecular weight. Isolation, purification and characterization of the amplified the markers DNA may be accomplished by excising or eluting the fragment from the gel (for example, see references Lawn et al., 1981; Goeddel et al., 1980), cloning the amplified product into a cloning site of a suitable vector, such as the pCRII vector (Invitrogen), sequencing the cloned insert and comparing the DNA sequence to the known sequence of the markers. The relative amounts of the markers mRNA and cDNA can then be determined.
(iii) Measurement of altered the markers protein levels in cell extracts or blood or other suitable biological sample, either qualitatively or quantitatively, for example by immunoassay, utilising immunointeractive molecules such as monoclonal antibodies.
   In one example, one may seek to detect the markers—immunointeractive molecule complex formation. For example, an antibody according to the invention, having a reporter molecule associated therewith, may be utilized in immunoassays. Such immunoassays include but are not limited to radioimmunoassays (RIAs), enzyme-linked immunosorbent assays (ELISAs) and immunochromatographic techniques (ICTs), Western blotting which are well known to those of skill in the art. For example, reference may be made to “Current Protocols in Immunology”, 1994 which discloses a variety of immunoassays which may be used in accordance with the present invention. Immunoassays may include competitive assays. It will be understood that the present invention encompasses qualitative and quantitative immunoassays.
   Suitable immunoassay techniques are described, for example, in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen. The antigen in this case is the markers or a fragment thereof.
   Two-site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilized first antibody.
An alternative method involves immobilizing the antigen in the biological sample and then exposing the immobilized antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.
From the foregoing, it will be appreciated that the reporter molecule associated with the antigen-binding molecule may include the following:—
   (a) direct attachment of the reporter molecule to the antibody;
   (b) indirect attachment of the reporter molecule to the antibody; i.e., attachment of the reporter molecule to another assay reagent which subsequently binds to the antibody; and
   (c) attachment to a subsequent reaction product of the antibody.
The reporter molecule may be selected from a group including a chromogen, a catalyst, an enzyme, a fluorochrome, a chemiluminescent molecule, a paramagnetic ion, a lanthanide ion such as Europium (Eu³⁴), a radioisotope including other nuclear tags and a direct visual label.
In the case of a direct visual label, use may be made of a colloidal metallic or non-metallic particle, a dye particle, an enzyme or a substrate, an organic polymer, a latex particle, a liposome, or other vesicle containing a signal producing substance and the like.
A large number of enzymes suitable for use as reporter molecules is disclosed in U.S. Pat. Nos. 4,366,241, 4,843,000, and 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.
Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al., International Publication No. WO 93/06121. Reference also may be made to the fluorochromes described in U.S. Pat. No. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.
In the case of an enzyme immunoassay, an enzyme is conjugated to the second antibody, generally by means of glutaraldehyde or periodate. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex, allowed to bind, and then the excess reagent washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.
Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules may also be employed.
(iv) The use of aptamers in screening for nucleic acid molecules or expression products
(v) Determining altered protein expression based on any suitable functional test, enzymatic test or immunological test in addition to those detailed in point (iii)—above.

As detailed above, any suitable technique may be utilized to detect the markers or their encoding nucleic acid molecule. The nature of the technique which is selected for use will largely determine the type of biological sample which is required for analysis. Such determinations are well within the scope of the person of skill in the art. Typical samples which one may seek to analyze are biopsy samples of the prostate or blood samples.

Another aspect of the present invention provides a diagnostic kit for assaying biological samples comprising an agent for detecting the marker proteins or encoding nucleic acid molecules and reagents useful for facilitating the detection by the agent in the first compartment. Further means may also be included, for example, to receive a biological sample. The agent may be any suitable detecting molecule.

The present invention is further described by reference to the following non-limiting examples.

### EXAMPLES

Levels of activin in normal patient sera in the following examples have been measured using a specific 2 site EIA as reported by Loria P. et al. in European Journal of Endocrinology (1998) 139 487-492. The results they reported were as follows:

TABLE 3
Serum concentrations of activin A in patients.

| | Women | | Men | | | Total | |
|---|---|---|---|---|---|---|---|
| Age Group | | Activin A | | Activin A | | | Activin A |
| (years) | n | (ng/ml) | n | (ng/ml) | p | n | (ng/ml |
| 20-30 | 18 | 0.05 ± 0.17 | 10 | 0.5260.10 | NS | 28 | 0.5160.15 |
| 30-40 | 20 | 0.55 ± 0.28 | 26 | 0.63 ± 0.10 | NS | 46 | 0.60 ± 0.20 |
| 40-50 | 50 | 0.77 ± 0.21 | 25 | 0.75 ± 0.14 | NS | 75 | 0.77 ± 0.19 |
| 50-60 | 31 | 0.58 ± 0.13 | 15 | 1.05 ± 0.17 | <0.001 | 46 | 0.73 ± 0.27 |
| 60-70 | 12 | 0.59 ± 0.10 | 14 | 1.11 ± 0.20 | <0.001 | 26 | 0.87 ± 0.31 |
| 70-80 | 20 | 0.67 ± 0.10 | 16 | 1.09 ± 0.18 | <0.001 | 36 | 0.86 ± 0.26 |
| Total | 151 | 0.64 ± 0.21 | 106 | 0.84 ± 0.26 | <0.001 | 257 | 0.73 ± 0.25 |

Data are expressed as means ±SD n, number of patients studied. Statistical analysis was performed by Student's t-test for unpaired data compared with the same age group of different sex.

While these authors used the same activin ELISA as used in the examples below, they used a different reference standard. To compare the above results to those detailed in these examples, it is necessary to apply a correction factor of ˜2.4 times higher than what the inventors measured. This is an important point in defining normal ranges and concentrations: these will vary from assay to assay. While the inventors have assigned numeric cutoffs with the assays described herein which indicate poor prognosis the skilled addressee will recognise that different assays will exhibit different numeric cutoff values.

For follistatin the normal range is also variable depending on the assay. A typical normal range would be <12 ng/ml or more conservatively <15 ng/ml.

1. Data from normal patients presented in the examples below: “normal” controls were all <12 ng/ml.

### EXAMPLE 1

### Serum Concentration of Activin and Follistatin are Elevated and Run in Parallel in Patients with Septicemia

Materials and Methods

As part of their routine clinical management, serial blood samples were collected from seven female and eight male patients of different ages who suffered from septic infections of different grades of severity. After completion of the clinical routine analyses, follistatin and activin were measured in the remnants of serum samples. Since the patients were critically ill, no extra blood samples were drawn for the purposes of this study. The samples were stored frozen at −20° C. until assayed. Since follistatin and activin serum levels increase with age [Wakatsuki et al., 1996 supra; Loria et al., 1998, supra], serum samples from age- and sex-matched healthy volunteers served as controls. All samples from diseased and healthy persons were treated in the same way.

Patients were categorized for septicemia according to the American College of Chest Physicians/Society of Critical Care Medicine (ACCP/SCCM) Consensus criteria (manifestation of two or more of the following clinical conditions: body temperature >38° C. or <36° C.; heart rate >90 beats/minute; respiratory rate >20 breaths/minute or PaCO₂ <32 mmHg; white blood cell count >12000 cells/mm³, <4000 cells/mm³, or >10% immature forms). For-twelve of the fifteen patients, the diagnosis of septicemia was proven by culture of the infectious organism from blood. In three cases the culture of the infectious organism failed due to the rapid implementation of antibiotic treatment.

Activin A concentrations in serum were measured using a specific ELISA which detects both follistatin-bound and free activin [Knight et al., 1996, supra], with the following modifications. The standard used was human recombinant (hr) activin A as described previously [McFarlane et al., 1996 supra]. The assay sensitivity was 0.1 ng/ml and the intra- and inter-assay coefficients of variation were 4.7% and 7.8% respectively. Serum samples were assayed against the standard diluted in 5% bovine serum albumin in phosphate-buffered saline (0.01 molecule/I). Follistatin concentrations in serum were measured with a radioimmunoassay validated for human follistatin as previously described [O'Connor et al., 1999 supra].

The standard employed was hrFS 288, but the assay crossreacts with hrFS 315 (35.9%). The assay sensitivity was 2.0 ng/ml and the intra- and interassay coefficients of variation were both <4.9%. The assay measures total follistatin (free and bound). Numbers of leukocytes, serum creatinine levels, and serum C-reactive protein levels were determined by clinical routine methods in the department of Clinical chemistry of the University of Gottingen.

Differences in the serum concentrations of follistatin and activin between septic patients and matched healthy volunteers were analyzed by paired t-test. Correlations between measured parameters were calculated with Pearson correlation. The software was Graph Pad Prism 3.0 (Graph Pad, San Diego, Calif. USA).

Results

Peak activin and follistatin serum concentrations of patients with septicemia were elevated compared with concentration in age- and sex-matched controls (Table 2). The median of the maximum activin concentration of septicaemic patients was 3.9-fold higher than the rnedian in healthy controls (P<0.01); the median of the maximum follistatin concentrations of septicaemic patients was 2.6-fold higher than the median of the follistatin concentrations in healthy controls (P<0.01). The magnitude of the activin and follistatin increase during septicemia varied among individuals and there was no close association between follistatin/activin serum concentrations and clinical outcome.

FIG. 10 depicts the individual profiles of serum activin and FS in the seven female (A) and eight male (B) patients and the corresponding serum levels of CRP, an indicator of inflammation. Most of the diagrams in FIG. 10 show that activin and FS serum levels track each other and follow changes in CRP levels. Overall, activin and FS concentrations were correlated with each other (r²=0.64). There was no apparent relationship between activin and FS serum concentrations and the number of leukocytes (r²=0.09). The parallel profiles of FS, activin, and CRP suggest a causal relationship between bacterial infection and elevated activin and FS serum levels. The observed increases in FS and activin serum concentrations during the inflammatory response are in accordance with observations in animal experiments, where interleukin-1β or lipopolysaccharide (LPS) injections caused significantly elevated FS and activin serum levels [Jones et al, 2000 supra; Klein et al, 1996 supra; Phillips et.al, 1996 supra]. The onset of septicemia is often unnoticed, whereas in most cases blood sampling commenced with obvious signs of the disease; this made it impossible to determine whether the serum concentration of activin, FS or CRP was the first to rise at the beginning of the infection.

The normal creatinine serum concentrations in most patients and the molecular size of the glycosylated follistatin (39-45 kDA) and activin (25 kDa) molecules make an altered renal function as the sole cause of increased serum concentrations in sepsis unlikely.

### EXAMPLE 2

Follistatin and activin levels were assessed over time in 8 patients as follows.

The methodology used corresponds to that described in Example 1.

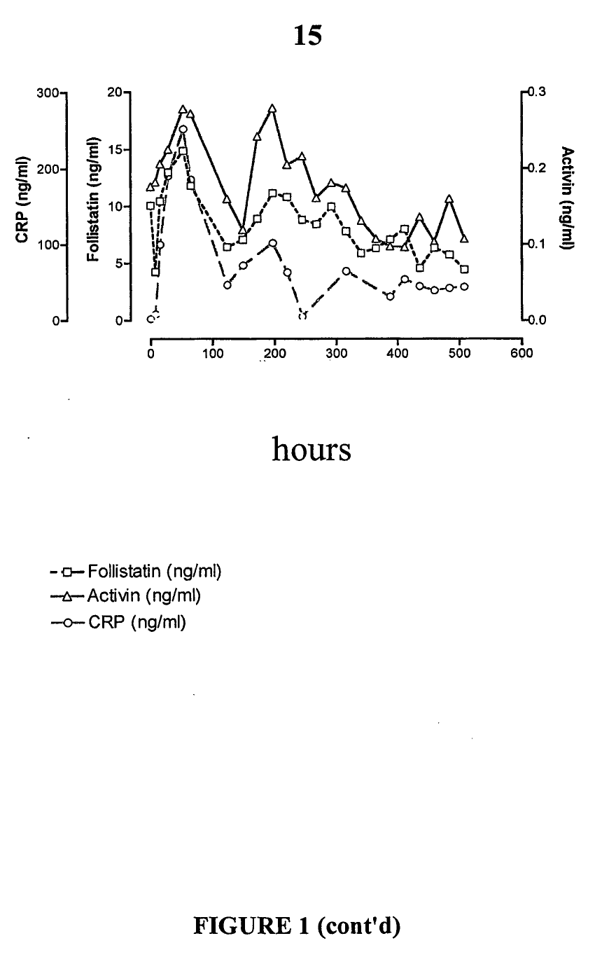

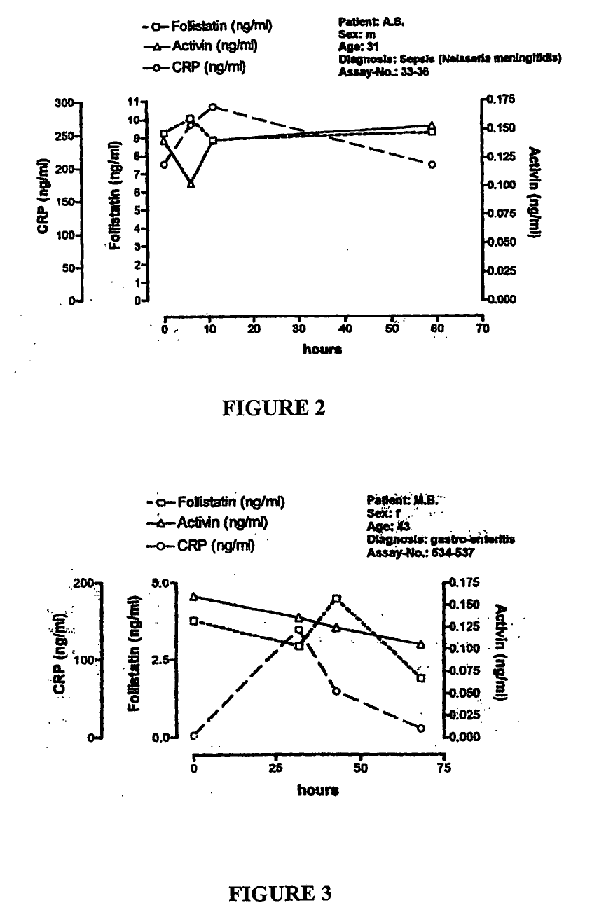

FIGS. 2 to 9 show the levels of activin, follistatin and C-reactive protein in these patients.

FIG. 2 shows a time course of activin, follistatin and C-reactive protein concentrations in serum of patient A.S. a 31 year old male diagnosed with Neisseria meningitidis meningitis and sepsis who subsequently died. For this patient serum activin levels ranged between 0.100 and 0.150 ng/ml while follistatin levels ranged between 9 and 10 ng/ml.

FIG. 3 shows a time course of a time course of activin, follistatin and C-reactive protein concentrations in serum of patient M.B. a 43 year old female diagnosed with gastroenteritis who recovered. For this patient serum activin levels ranged between 0.100 and 0.150 ng/ml while follistatin levels ranged between 2 and 4 ng/ml.

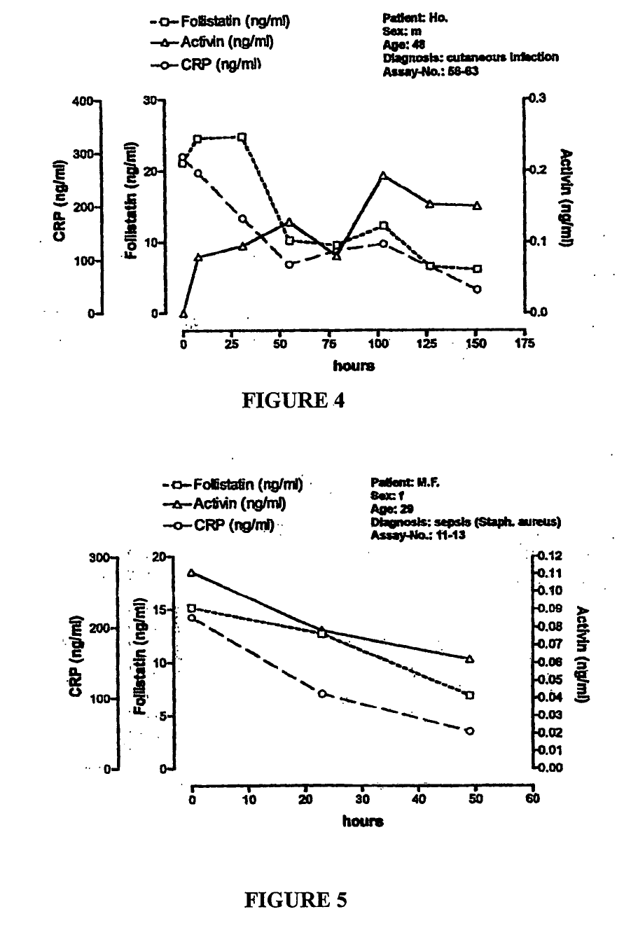

FIG. 4 shows a time course of activin, follistatin and C-reactive protein concentrations in serum of patient Ho a 48 year old male diagnosed with a cutaneous infection. The patient recovered. For this patient serum activin levels ranged between 0 and 0.2 ng/ml while follistatin levels ranged between 9 and 25 ng/ml.

FIG. 5 shows a time course of activin, follistatin and C-reactive protein concentrations in serum of patient M.F. a 29 year old female diagnosed with Staphylococcus aureus sepsis who recovered. For this patient serum activin levels ranged between 0.060 and 0.105 ng/ml while follistatin levels ranged between 9 and 15 ng/ml.

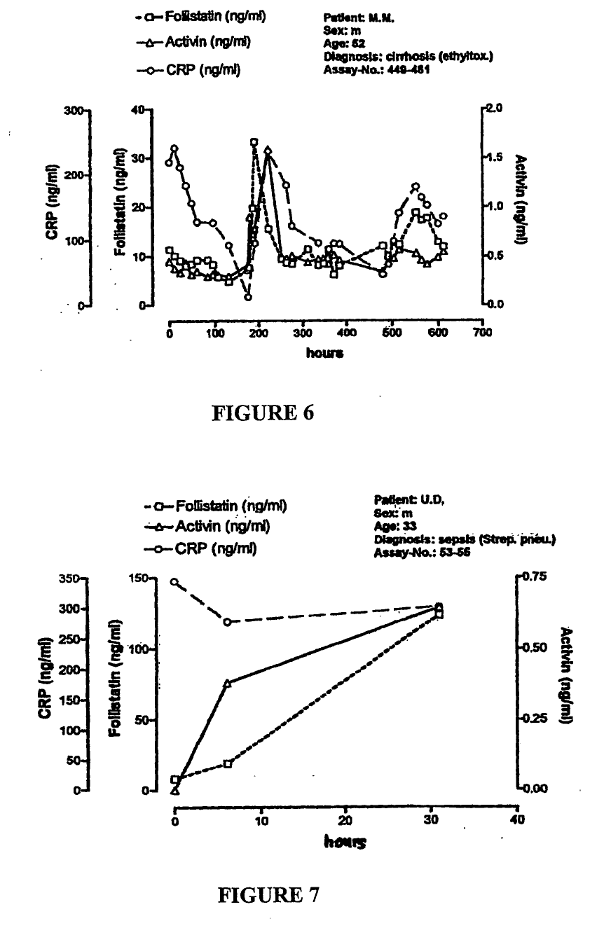

FIG. 6 shows a time course of activin, follistatin and C-reactive protein concentrations in serum of patient M.M. a 52 year old male diagnosed with cirrhosis who subsequently died. For this patient serum activin levels ranged between 0.25 and 0.50 ng/ml while follistatin levels ranged between 5 and 32 ng/ml.

FIG. 7 shows a time course of activin, follistatin and C-reactive protein concentrations in serum of patient U.D. a 33 year old male diagnosed with Streptococcus pneumoniae sepsis who subsequently died. For this patient serum activin levels ranged between 0 and 0.68 ng/ml while follistatin levels ranged between 5 and 125 ng/ml.

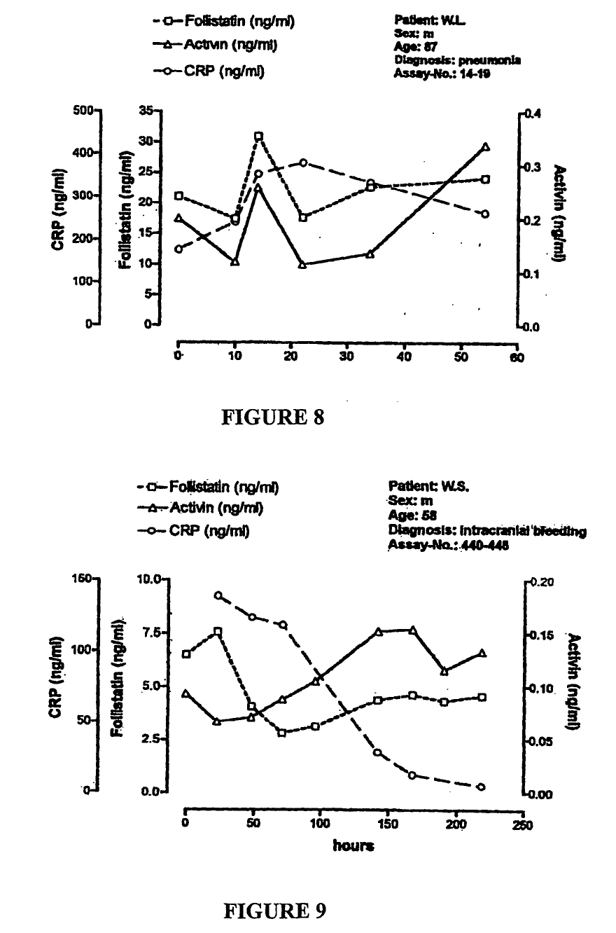

FIG. 8 shows a time course of activin, follistatin and C-reactive protein concentrations in serum of patient W/L. an 87 year old male diagnosed with pneumonia who subsequently died. For this patient serum activin levels ranged between 0.11 and 0.33 ng/ml while follistatin levels ranged between 17 and 32 ng/ml.

FIG. 9 shows a time course of activin, follistatin and C-reactive protein concentrations in serum of patient W.S. a 58 year old male diagnosed with intracranial bleeding. For this patient serum activin levels ranged between 0.07 and 0.15 ng/ml while follistatin levels ranged between 2.5 and 7.5 ng/ml.

Typically patients in whom activin levels remained below 0.3 ng/ml and follistatin levels remained below 20 ng/ml recovered whereas patients with higher levels dies.

### EXAMPLE 3

Follistatin and activin levels are assessed over time in a group of patients with meningitis and other brain disorders.

The methodology is as described in Example 1. For measuring activin A in CSF samples, the standard diluent used is 0.05% BSA in PBS to match the protein concentration in the samples. A 20% solution of BSA in PBS (25 μL) is added to the wells before the addition of CSF samples as this is found to enhance the reproducibility of the assay.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Billestrup N, Gonzalez-Manchon C, Potter E & Vale W. Inhibition of somatotroph growth and growth hormone biosynthesis by activin in vitro. Molecular Endocrinology 1990 4 356-362.

de Winter J P, ten Dijke P, de Vries C J, van Achterberg T A, Sugino H, de Waele P et al. Follistatins neutralize activin bioactivity by inhibition of activin binding to its type II receptors. Molecular and Cellular Endocrinology 1996 116 105-114.

Demura R, Suzuki T, Tajina S, Mitsuhashi S, Odagiri E, Demura H et al. Human plasma free activin and inhibin levels during the menstrual cycle. Journal of Clinical Endocrinology and Metabolism 1993 76 1080-1082.

Demura R, Suzuki T, Tajima S, Mitsuhashi S, Odagiri E, Eto Yet al. Competitive protein binding assay for activin A/EDF using follistatin determination of activin levels in human plasma. Biochemical and Biophysical Research Communications 1992 185 1148-1154.

Erämaa M, Hurme M, Stenman U H & Ritvos O. Activin A/erythroid differentiation factor is induced during human monocyte activation. Journal of Experimental Medicine 1992 176 1449-1452.

Erlich, 1989

Fodor et al., 1991

Gilfillan C P & Robertson D M. Development and validation of a radioimmunoassay for follistatin in human serum. Clinical Endocrinology 1994 41 453-461.

Goeddel et al., 1980

Hashimoto O, Nakamura T, Shoji H, Shimasaki S, Hayashi Y & Sugino H. A novel role of follistatin, an activin-binding protein, in the inhibition of activin action in rat pituitary cells. Endocytotic degradation of activin and its acceleration by follistatin associated with cell-surface heparan sulfate. Journal of Biological Chemistry 1997 272 13835-13842.

Hübner G & Werner S. Serum growth factors and proinflammatory cytokines are potent inducers of activin expression in cultured fibroblasts and keratinocytes. Experimental Cell Research 1996 228 106-113.

Hübner G, Brauchle M, Gregor M & Werner S. Activin A: a novel player and inflammatory marker in inflammatory bowel disease? Laboratory Investigation 1997 77 311-318.

Hübner G, Hu Q, Smola H & Werner S. Strong induction of activin expression after injury suggests an important role of activin in wound repair. Developmental Biology 1996 173 490-498.

Jones K L, Brauman J N, Groome N P, de Kretser D M & Phillips D J. Activin A release into the circulation is an early event in systemic inflammation and precedes the release of follistatin. Endocrinology 2000 141 1905-1908.

Jones K L, Brauman J N, Groome N P, de Kretser D M and Phillips D J, Activin A release into the circulation is an early event in systemic inflammation and precedes the release of follistatin. Endocrinology 2000 141 1905-1908.

Kazal et al., 1996

Keelan J A, Zhou R L & Mitchell M D. Activin A exerts both pro- and anti-inflammatory effects on human term gestational tissues. Placenta 2000 21 38-43.

Khoury R H, Wang Q F, Crowley W F Jr, Hall J E, Schneyer A L, Toth T et al. Serum follistatin levels in women: evidence against an endocrine function of ovarian follistatin. Journal of Clinical Endocrinology and Metabolism 1995 80 1361-1368.

Kitaoka M, Kojima I & Ogata E. Activin-A: a modulator of multiple types of anterior pituitary cells. Biochemical and Biophysical Research Communications 1988 157 48-54.

Klein R, Clarke I J, Hedger M P and Robertson D M, Plasma follistatin concentrations increase following lipopolysaccharide administration in sheep. Clin Exp Pharmacol Physiol 1996 23 754-755.

Knight P G, Muttukrishna S & Groome N P, Development and application of a two-site enzyme immunoassay for the determination of ‘total’ activin-A concentrations in serum and follicular fluid. J Endocrinol 1996 148 267-279

Knight P G, Muttukrishna S & Groome N P. Development and application of a two-site enzyme immunoassay for the determination of ‘total’ activin-A concentrations in serum and follicular fluid. Journal of Endocrinology 1996 148 267-279.

Lawn et al., 1981

Loria P, Petraglia F, Concari M, Bertolotti M, Martella P, Luisi S et al. Influence of age and sex on serum concentrations of total dimeric activin A. European Journal of Endocrinology 1998 139 487-492.

Loria P, Petraglia F, Concari M, Bertolotti M, Martella P, Luisi S, et al. Influence of age and sex on serum concentrations of total dimeric activin A. Eur J Endocrinol 1998 139 487-492

McFarlane J R, Foulds L M, Pisciotta A, Robertson D M & de Kretser D M. Measurement of activin in biological fluids by radioimmunoassay, utilizing dissociating agents to remove the interference of follistatin. European Journal of Endocrinology 1996 134 481-489.

Meunier H, Rivier C, Evans R M & Vale W. Gonadal and extragonadal expression of inhibin a, b A, and b B subunits in various tissues predicts diverse functions. PNAS 1988 85 247-251.

Michel U, Albiston A & Findlay J K. Rat follistatin: gonadal and extragonadal expression and evidence for alternative splicing. Biochemical and Biophysical Research Communications 1990 173 401-407.

Michel U, Esselmann J & Nieschlag E. Expression of follistatin messenger ribonucleic acid in Sertoli cell-enriched cultures: regulation by epidermal growth factor and protein kinase C-dependent pathway but not by follicle-stimulating hormone and protein kinase A-dependent pathway. Acta Endocrinologica 1993 129 525-531.

Michel U, Schneider O, Kirchhof C, Meisel S, Smirnov A, Wiltfang J et al. Production of follistatin in porcine endothelial cells: differential regulation by bacterial compounds and the synthetic glucocorticoid RU 28362. Endocrinology 1996 137 4925-4934.

Mohan A, Asselin J, Sargent I L, Groome N P & Muttukrishna S. Effect of cytokines and growth factors on the secretion of inhibin A, activin A and follistatin by term placental villous trophoblasts in culture. European Journal of Endocrinology 2001 145 505-511.

Moore, A., Basilion, J., Chiocca, E., and Weissleder, R., 1988, BBA, 1402:239-249

Mullis et al., 1987

O'Connor A E, McFarlane J R, Hayward S, Yohkaichiya T, Groome N P and de Kretser D M, Serum activin A and follistatin concentrations during human pregnancy: a cross-sectional and longitudinal study. Hum Reprod 1999 14 827-832

Phillips D J, de Kretser D M, Pfeffer A, Chie W N & Moore L G. Follistatin has a biphasic response but follicle-stimulating hormone is unchanged during an inflammatory episode in growing lambs. Journal of Endocrinology 1998 156 77-82.

Phillips D J, Hedger M P, McFarlane Jr, Klein R, Clarke I J, Tilbrook A J, et al. Follistatin concentrations in male sheep increase following sham castration/castration or injection of interleukin-1 beta. J Endocrinol 1996 151 119-124.

Phillips D J. New developments in the biology of inhibins, activins and follistatins. Trends in Endocrinology and Metabolism 2001 12 94-96.

Sakai R, Shiozaki M, Tabuchi M & Eto Y. The measurement of activin/EDF in mouse serum: evidence for extragonadal production. Biochemical and Biophysical Research Communications 1992 188 921-926.

Sakamoto Y, Shintani Y, Harada K, Abe M, Shitsukawa K & Saito S. Determination of free follistatin levels in sera of normal subjects and patients with various diseases. European Journal of Endocrinology 1996 135 345-351.

Schneider O, Nau R & Michel U. Comparative analysis of follistatin-, activin beta A- and activin beta B-mRNA steady-state levels in diverse porcine tissues by multiplex S1 nuclease analysis. European Journal of Endocrinology 2000 142 537-544.

Shao L E, Frigon N L Jr, Yu A, Palyash J & Yu J. Contrasting effects of inflammatory cytokines and glucocorticoids on the production of activin A in human marrow stromal cells and their implications. Cytokine 1998 10 227-235.

Tilbrook A J, de Kretser D M, Dunshea F R, Klein R, Robertson D M, Clarke I J et al. The testis is not the major source of circulating follistatin in the ram. Journal of Endocrinology 1996 149 55-63.

Vale et al. (1990) In peptide growth factors and their receptors : Handbook of Experimental Physiology, Vol. 95, Eds. Sporn & Roberts, Springer-Verlag, Berlin pp211-248]

Vale W, Rivier J, Vaughan J, McClintock R, Corrigan A, Woo W et al. Purification and characterization of an FSH releasing protein from porcine ovarian follicular fluid. Nature 1986 321 776-779.

Wakatsuki M, Shintani Y, Abe M, Liu Z H, Shitsukawa K & Saito S. Immunoradiometric assay for follistatin: serum immunoreactive follistatin levels in normal adults and pregnant women. J Clin Endocrinol Metab 1996 81 630-634

Wakatsuki M, Shintani Y, Abe M, Liu Z H, Shitsukawa K & Saito S. Immunoradiometric assay for follistatin: serum immunoreactive follistatin levels in normal adults and pregnant women. Journal of Clinical Endocrinology and Metabolism 1996 81 630-634.

Wedemeyer, N., Potter, T., Wetzlich, S. and Gohde, W. 2002, Clinical Chemistry 48:9 1398-1405

Weissleder, R., Moore, A., Ph.D., Mahmood-Bhorade, U., Benveniste, H., Chiocca, E. A., Basilion, J. P. 2000, Nature Medicine, 6:351-355

Yu E W, Dolter K E, Shao L E & Yu J. Suppression of IL-6 biological activities by activin A and implications for inflammatory arthropathies. Clinical and Experimental Immunology 1998 112 126-132.

Yu J, Shao L E, Frigon N L Jr, Lofgren J & Schwall R. Induced expression of the new cytokine, activin A, in human monocytes: inhibition by glucocorticoids and retinoic acid. Immunology 1996 88 368-374.

TABLE 1

| | | | | Septicemia (ng/ml) | | Control (ng/ml) | | |
|---|---|---|---|---|---|---|---|---|
| No. | Diagnosis | Age | Sex | Activin/Follistatin | | Activin/Follistatin | | Outcome |
| 1 | Sepsis (Staph. aureua) | 64 | f | 0.42 | 25.77 | 0.15 | 4.84 | Deceased |
| 2 | Sepsis & meningitis (Strept. pneu.) | 72 | f | 0.65 | 14.48 | 0.18 | 10.4 | Cured |
| 3 | Sepsis & meningitis (Neisseria meningitidis) | 42 | f | 0.15 | 5.27 | 0.07 | 5.7 | Cured |
| 4 | Sepsis & meningitis (Strept. pneu.) | 56 | f | 0.59 | 15.00 | 0.1 | 5.6 | Deceased |
| 5 | Sepsis & encephalitis & endocarditis (coagulase- | 84 | f | 1.97 | 39.64 | 0.18 | 10.4 | Deceased |
| | negative Steph.) | | | | | | | |
| 6 | Sepsis (E. coli) | 67 | f | 0.61 | 42.28 | 0.16 | 5.7 | Deceased |
| 7 | Sepsis (Neisseria meningitidis) | 33 | f | 2.31 | 76.14 | 0.08 | 5.1 | Deceased |
| 8 | Sepsis | 66 | m | 0.62 | 16.82 | 0.13 | 3.88 | Deceased |
| 9 | Sepsis & meningitis (Strep. pneu.) | 37 | | 0.19 | 16.39 | 0.11 | 5.75 | Cured |
| 10 | Sepsis (Staph. aureus) | 70 | m | 0.81 | 21.9 | 0.15 | 6.48 | Deceased |
| 11 | Sepsis & meningitis (Neisseria meningitidis) | 46 | m | 0.18 | 5.26 | 0.13 | 5.34 | Cured |
| 12 | Sepsis (coagulase-negative Staph.) | 75 | m | 0.19 | 15.01 | 0.18 | 11.3 | Cured |
| 13 | Sepsis | 62 | m | 0.43 | 9.97 | 0.17 | 10.84 | Deceased |
| 14 | Sepsis & pneumonia | 76 | m | 0.28 | 12.47 | 0.18 | 9.36 | Cured |
| 15 | Sepsis (Staph. Epidermidis) | 71 | m | 0.28 | 14.88 | 0.17 | 7.71 | Cured |
| | Median | | | 0.59 | 15 | 0.15 | 5.75 | |

Table 1 shows a comparison of activin and follistatin serum levels of patients with septicemia and healthy age and sex-matched controls. The concentration given for septic patients i the peak level observed across multiple samples.
Staph., Staphylococcus;
Strept., Streptococcus;
pneu., pneumoniae;
m, male;
f, female;
control versus septicemia: p = 0.004 for activin concentrations and p = 0.009 for follistatin concentrations (paired Wilcoxon rank test).
*patient died soon after discharge from clinic. With the exception of one patient, in all deceased cases there is a “significant” increase in follistatin in sepsis compared to controls (increase in follistatin is normally associated also with an increase in activin). Conversely, where the increase in follistatin is less than approximately two-fold compared with controls, an association with a positive outcome can be detected.

TABLE 2
Comparison of activin and FS serum concentrations from patients with
septicemia and sex- and age-matched healthy controls

| | | | | Septicemia | | Control | | |
|---|---|---|---|---|---|---|---|---|
| | | | | (ng/ml) | | (ng/ml) | | |
| Number | Diagnosis | Age | Sex | Activin | FS | Activin | FS | Outcome |
| 1 | Sepsis (Staph. aureus) | 64 | f | 0.42 | 25.77 | 0.15 | 4.84 | Deceased |
| 2 | Sepsis & meningitis (Strep. pneu.) | 72 | f | 0.65 | 14.48 | 0.18 | 10.4 | Cured |
| 3 | Sepsis & meningitis (Neisseria meningitidis) | 42 | f | 0.15 | 5.27 | 0.07 | 5.7 | Cured |
| 4 | Sepsis & meningitis (Strep. pneu.) | 56 | f | 0.59 | 15.00 | 0.1 | 5.6 | Deceased |
| 5 | Sepsis & encephalitis & endocarditis | 84 | f | 1.97 | 39.64 | 0.18 | 10.4 | Deceased* |
| | (coagulase-negative Staph.) | | | | | | | |
| 6 | Sepsis (E. coli) | 67 | f | 0.61 | 42.28 | 0.16 | 5.7 | Deceased |
| 7 | Sepsis (Neisseria meningitidis) | 33 | f | 2.31 | 76.14 | 0.08 | 5.1 | Deceased |
| 8 | Sepsis | 66 | m | 0.62 | 16.82 | 0.13 | 3.88 | Deceased |
| 9 | Sepsis & meningitis (Strep. pneu.) | 37 | m | 0.19 | 16.39 | 0.11 | 5.75 | Cured |
| 10 | Sepsis (Staph. aureus) | 70 | m | 0.81 | 21.9 | 0.15 | 6.48 | Deceased |
| 11 | Sepsis & meningitis (Neisseria meningitidis) | 46 | m | 0.18 | 5.26 | 0.13 | 5.34 | Cured |
| 12 | Sepsis (coagulase-negative Staph.) | 75 | m | 0.19 | 15.01 | 0.18 | 11.3 | Cured |
| 13 | Sepsis | 62 | m | 0.43 | 9.97 | 0.17 | 10.84 | Deceased |
| 14 | Sepsis & pneumonia | 76 | m | 0.28 | 12.47 | 0.18 | 9.36 | Cured |
| 15 | Sepsis (Staph. epidermidis) | 71 | m | 0.28 | 14.88 | 0.17 | 7.71 | Cured |
| | Median | | | 0.59 | 15 | 0.15 | 5.75 | |

Staph., Staphylococcus;
Strep., Streptococcus;
pneu., pneumoniae;
E. coli, Escherichia coli;
m, male;
f, female.
Control vs septicemia: P = 0.004 for activin concentrations and P = 0.009 for FS concentrations (paired Wilcoxon rank test).
*Patient died soon after discharge from clinic

1. A method for detecting the onset or a predisposition to the onset of an inflammatory response in a mammal, said method comprising screening for the level of one or both of activin or follistatin protein and/or gene expression in said mammal wherein an increase in the level of said protein and/or gene expression is indicative of an inflammatory response.

2. The method according to claim 1 wherein said activin is activin A.

3. The method according to claim 2 wherein said inflammatory response is a local inflammatory response.

4. The method according to claim 2 wherein said inflammatory response is a systemic inflammatory response.

5. The method according to claim 3 or 4 wherein said inflammatory response is related to septic shock, toxic shock, sepsis, septicaemia, pancreatitis, appendicitis, meningitis, hepatic response to toxins or viruses, angiogenesis, psoriasis, neural protection, atherosclerosis, renal tubular necrosis, wound healing, traumatic injury, surgery or burns.

6. The method according to claim 5 wherein said inflammatory response is acute.

7. The method according to claim 4 wherein said systemic inflammatory response is related to systemic inflammatory response syndrome such as sepsis, septic shock, toxic shock, septicaemia, tissue trauma, meningitis or appendicitis.

8. The method according to claim 7 wherein said inflammatory response is acute.

9. A method for monitoring the progression of an inflammatory response in a mammal, said method comprising screening for modulation of the level of one or both of activin or follistatin protein and/or gene expression in said mammal wherein an increase in the level of said protein and/or gene expression relative to a previously obtained level is indicative of the maintenance or worsening of said response and a decrease in said level is indicative of an improvement in said inflammatory response.

10. The method according to claim 9 wherein said activin is activin A.

11. The method according to claim 10 wherein said inflammatory response is a local inflammatory response.

12. The method according to claim 10 wherein said inflammatory response is a systemic inflammatory response.

13. The method according to claim 3 or 4 wherein said inflammatory response is related to septic shock, toxic shock, sepsis, septicaemia, appendicitis, pancreatitis, meningitis, hepatic response to toxins or viruses, angiogenesis, psoriasis, neural protection, atherosclerosis, renal tubular necrosis, wound healing, traumatic injury, surgery or burns.

14. The method according to claim 13 wherein said inflammatory response is acute.

15. The method according to claim 12 wherein said systemic inflammatory response is related to systemic inflammatory response syndrome such as sepsis, septicaemia, tissue trauma, meningitis or appendicitis.

16. The method according to claim 15 wherein said inflammatory response is acute.

17. A method for assessing the severity of an inflammatory response in a mammal, said method comprising quantitatively screening for the level of one or both of activin or follistatin protein and/or gene expression wherein the degree of increase in the level of said protein and/or gene expression is indicative of the severity of said inflammatory response.

18. The method according to claim 17 wherein said activin is activin A.

19. The method according to claim 18 wherein said inflammatory response is a local inflammatory response.

20. The method according to claim 18 wherein said inflammatory response is a systemic inflammatory response.

21. The method according to claim 19 or 20 wherein said inflammatory response is related to septic shock, sepsis, toxic shock, septicaemia, appendicitis, pancreatitis, meningitis, hepatic response to toxins or viruses, angiogenesis, psoriasis, neural protection, atherosclerosis, renal tubular necrosis, wound healing, traumatic injury, surgery or burns.

22. The method according to claim 21 wherein said inflammatory response is acute.

23. The method according to claim 20 wherein said systemic inflammatory response is related to systemic inflammatory response syndrome such as sepsis, toxic shock, septic shock, septicaemia, tissue trauma, meningitis or appendicitis.

24. The method according to claim 23 wherein said inflammatory response is acute.

25. The method according to any one of claims 17-24 wherein the greater the severity of said inflammatory response, the poorer the prognosis for the subject mammal.

26. The method according to claim 25 wherein said acute inflammatory response is sepsis.

27. The method according to claim 26 wherein a level of activin A and/or follistatin protein is at least about 2 times higher than levels within the normal range is indicative of a poor prognosis for said mammal.

28. The method according to claim 27 wherein said level is at least about three times higher than levels within the normal range.

29. The method according to claim 28 wherein said activin A is greater than 0.3 ng/ml or a 24 hour period and/or the level of follistatin is greater than 20 ng/ml over a 24 hour period.

30. The method according to any one of claims 27 to 29 wherein said poor prognosis is death.

31. A method for detecting the onset or a predisposition to the onset of a condition characterised by an inflammatory response in a mammal, said method comprising screening for the level of one or both of activin or follistatin protein and/or gene expression in said mammal where an increase in the level of said protein and/or gene expression is indicative of the onset or predisposition to the onset of said condition.

32. A method for monitoring the progression of a condition characterised by an inflammatory response in a mammal, said method comprising screening for modulation of the level of one or both of activin or follistatin proteins and/or gene expression in said mammal wherein an increase in the level of said protein and/or gene expression relative to a previously obtained level is indicative of the maintenance or worsening of said condition and a decrease in said level is indicative of an improvement in said condition.

33. A method for assessing the severity of a condition characterised by an inflammatory response in a mammal, said method comprising quantitatively screening for the level of one or both of activin or follistatin protein and/or gene expression in said mammal wherein the degree of increase in the level of said protein and/or gene expression is indicative of the severity of said condition.

34. The method according to any one of claims 31 to 33 wherein said activin is activin A.

35. The method according to claim 34 wherein said inflammatory response is a local inflammatory response.

36. The method according to claim 34 wherein said inflammatory response is a systemic inflammatory response.

37. The method according to claim 35 or 36 wherein said condition is septic shock, sepsis, toxic shock, septicaemia, appendicitis, pancreatitis, meningitis, hepatic response to toxins or viruses, angiogenesis, psoriasis, neural protection, atherosclerosis, renal tubular necrosis, wound healing, traumatic injury, surgery or burns.

38. The method according to claim 37 wherein said inflammatory response is acute.

39. The method according to claim 36 wherein said condition is systemic inflammatory distress syndrome such as sepsis, septic shock, toxic shock, septicaemia, tissue trauma, meningitis or appendicitis.

40. The method according to claim 39 wherein said inflammatory response is acute.

41. The method according to claim 33 wherein the greater the severity of said inflammatory response, the poorer the prognosis for the subject mammal.

42. The method according to claim 41 wherein said acute inflammatory response is sepsis.

43. The method according to claim 42 wherein a level of activin A and/or follistatin protein is at least about 2 times higher than levels within the normal range is indicative of a poor prognosis for said mammal.

44. The method according to claim 43 wherein said level is at least about three times higher than levels within the normal range.

45. The method according to claim 44 wherein said activin A is greater than 0.3 ng/ml or a 24 hour period and/or the level of follistatin is greater than 20 ng/ml over a 24 hour period.

46. The method according to any one of claims 43 to 45 wherein said poor prognosis is death.

47. The method according to any one of claims 1-46 wherein said screening is directed to activin and/or follistatin protein.

48. The method according to any one of claims 4, 7, 8, 12, 15, 16, 20, 23, 24, 36, 39 or 40 wherein said systemic inflammatory response is assessed based on analysis of peripheral levels of activin A and/or follistatin protein.

49. The method according to claim 48 wherein said peripheral levels of activin A and/or follistatin are blood levels.

50. The method according to claim 49 wherein said blood levels are assessed based on the analysis of a sample of blood or component derived therefrom.

51. The method according to any one of claims 1-50 wherein both activin and follistatin levels are assessed.

52. The method according to any one of claims 1 to 51 wherein said mammal is a human.
